(19) United States
(12) Patent Application Publication
(10) Pub. No.: US 2020/0001096 A1
(43) Pub. Date: Jan. 2, 2020

(54) Systems and Methods for Thermal Stimulation of the Spinal Cord

(71) Applicant: Boston Scientific Neuromodulation Corporation, Valencia, CA (US)

(72) Inventors: Tianhe Zhang, Studio City, CA (US); Bradley Hershey, Carrollton, TX (US); Rosana Esteller, Santa Clarita, CA (US); Marom Bikson, New York, NY (US)

(21) Appl. No.: 16/443,541

(22) Filed: Jun. 17, 2019

Related U.S. Application Data
(60) Provisional application No. 62/692,976, filed on Jul. 2, 2018.

(51) Int. Cl. A61N 1/372; A61F 7/12; A61N 7/02; A61N 1/40; A61N 1/05; A61N 1/36

(52) U.S. Cl. CPC A61N 1/37247; A61F 7/12; A61N 7/02; A61N 1/406; A61F 2007/0093; A61N 1/36062; A61N 1/36139; A61F 2007/126; A61N 1/0551

(57) ABSTRACT

Methods and systems for providing dosed and calibrated thermal stimulation using an implantable stimulation device are disclosed. Aspects of the disclosure provide bioheat models based on physiological and thermal properties of target anatomy and thermopole algorithms that interact with the bioheat models to derive thermal stimulation parameters for providing dosed and calibrated thermal stimulation. Also, graphical user interfaces (GUIs) are disclosed for configuring and targeting heat delivery into specific targets.

### CROSS REFERENCE TO RELATED APPLICATIONS

This is a non-provisional application of U.S. Provisional Patent Application Ser. No. 62/692,976, filed Jul. 2, 2018, which is incorporated by reference, and to which priority to claimed.

### FIELD OF THE INVENTION

The present invention relates generally to medical device systems, and more particularly to pulse generator systems operable to measure spinal cord potentials (SCPs).

### INTRODUCTION

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and Deep Brain Stimulators (DBS) to treat motor and other neurological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any Implantable Medical Device (IPG) or in any IPG system, such as in a Deep Brain Stimulation (DBS) system as disclosed in U.S. Pat. No. 9,119,964.

An SCS system typically includes an Implantable Pulse Generator (IPG) 10 shown in plan and cross-sectional views in FIGS. 1A and 1B. The IPG 10 includes a biocompatible device case 30 is configured for implantation in a patient's tissue that holds the circuitry and battery 36 (FIG. 1B) necessary for the IPG to function. The IPG 10 is coupled to electrodes 16 via one or more electrode leads 14 that form an electrode array 12. The electrodes 16 are configured to contact a patient's tissue and are carried on a flexible body 18, which also houses the individual lead wires 20 coupled to each electrode 16. The lead wires 20 are also coupled to proximal contacts 22, which can be inserted into lead connectors 24 fixed in a header 28 on the IPG 10, which header can comprise an epoxy for example. Once inserted, the proximal contacts 22 connect to header contacts 26 in the lead connectors 24, which are in turn coupled by electrode feedthrough pins 34 through an electrode feedthrough 32 to circuitry within the case 30 (connection not shown).

In the illustrated IPG 10, there are thirty-two lead electrodes (E1-E32) split between four leads 14 (referred to as percutaneous leads), with the header 28 containing a 2×2 array of lead connectors 24 to receive the leads' proximal ends. However, the number of leads and electrodes in an IPG is application specific and therefore can vary. In a SCS application, the electrode leads 14 are typically implanted proximate to the dura in a patient's spinal cord, and when a four-lead IPG 10 is used, these leads can be split with two on each of the right and left sides. The proximal contacts 22 are tunneled through the patient's tissue to a distant location such as the buttocks where the IPG case 30 is implanted, at which point they are coupled to the lead connectors 24. As also shown in FIG. 1A, one or more flat paddle leads 15 can also be used with IPG 10, and in the example shown thirty-two electrodes 16 are positioned on one of the generally flat surfaces of the head 17 of the paddle lead, which surface would face the dura when implanted. In other IPG examples designed for implantation directly at a site requiring stimulation, the IPG can be lead-less, having electrodes 16 instead carried by the case of the IPG for contacting the patient's tissue.

As shown in the cross section of FIG. 1B, the IPG 10 includes a printed circuit board (PCB) 40. Electrically coupled to the PCB 40 are the battery 36, which in this example is rechargeable; other circuitry 46 coupled to top and/or bottom surfaces of the PCB 40, including a microcontroller or other control circuitry necessary for IPG operation; a telemetry antenna—42a and/or 42b—for wirelessly communicating data with an external controller 50 (FIG. 2); a charging coil 44 for wirelessly receiving a magnetic charging field from an external charger (not shown) for recharging the battery 36; and the electrode feedthrough pins 34 (connection to circuitry not shown). If battery 36 is permanent and not rechargeable, charging coil 44 would be unnecessary.

The IPG 10 also includes one or more antennas 42a and 42b for transcutaneously communicating with external programming devices, such as a patient external controller 50 (FIG. 2), or a clinician programmer 90 (FIG. 3). Antennas 42a and 42b are different in shape and in the electromagnetic fields they employ. Telemetry antenna 42a comprises a coil, which can bi-directionally communicate with an external device via a magnetic induction communication link. Telemetry antenna 42b comprises a short-range Radio-Frequency (RF) antenna that operates in accordance with a short-range RF communication standard, such as Bluetooth, BLE, NFC, Zigbee, WiFi (802.11x), and the Medical Implant Communication Service (MICS) or the Medical Device Radiocommunications Service (MDRS).

Implantation of IPG 10 in a patient is normally a multi-step process, as explained with reference to FIG. 3. A first step involves implantation of the distal ends of the lead(s) 14 or 15 with the electrodes 16 into the spinal column 60 of the patient through a temporary incision 62 in the patient's tissue 5. (Only two leads 14 with sixteen total electrodes 16 are shown in FIG. 3 for simplicity). The proximal ends of the leads 14 or 15 including the proximal contacts 22 extend externally from the incision 62 (i.e., outside the patient), and are ultimately connected to an External Trial Stimulator (ETS) 70. The ETS 70 is used during a trial stimulation phase to provide stimulation to the patient, which may last for two or so weeks for example. To facilitate the connection between the leads 14 or 15 and the ETS 70, ETS extender cables 80 may be used that include receptacles 82 (similar to the lead connectors 24 in the IPG 10) for receiving the proximal contacts 22 of leads 14 or 15, and connectors 84 for meeting with ports 72 on the ETS 70, thus allowing the ETS 70 to communicate with each electrode 16 individually. Once connected to the leads 14 or 15, the ETS 70 can then be affixed to the patient in a convenient fashion for the duration of the trial stimulation phase, such as by placing the ETS 70 into a belt worn by the patient (not shown). ETS 70 includes a housing 73 for its control circuitry, antenna, etc., which housing 73 is not configured for implantation in a patient's tissue.

The ETS 70 essentially mimics operation of the IPG 10 to provide stimulation to the implanted electrodes 16, and thus includes contains a battery within its housing along with stimulation and communication circuitry like that provided in the IPG 10. Thus, the ETS 70 allows the effectiveness of stimulation therapy to be verified for the patient, such as whether therapy has alleviated the patient's symptoms (e.g., pain). Trial stimulation using the ETS 70 further allows for the determination of stimulation program(s) that seems promising for the patient to use once the IPG 10 is later implanted into the patient. A stimulation program may include stimulation parameters that specify for example: which of the electrodes 16 are to be active and used to issue stimulation pulses; the polarity of those active electrodes (whether they are to act as anodes or cathodes); the current or voltage amplitude (A) of the stimulation pulses; the pulse width (PW) of the stimulation pulses; the frequency (f) of the stimulation pulses; the duty cycle (DC) of the stimulation pulses (i.e., the percentage of time that the pulses are asserted relative to the period of the pulses) the shape of the stimulation waveform (e.g., one or more square pulses, one or more ramped pulses, one or more sinusoidal pulses, or even non-pulse-based waveforms, etc.); and other parameters related to issuing a burst of pulses, such as the number of pulses; etc.

The stimulation program executed by the ETS 70 can be provided or adjusted via a wired or wireless link 92 (wireless shown) from a clinician programmer 90. As shown, the clinician programmer 90 comprises a computer-type device, and may communicate wirelessly with the ETS 70 via link 92, which link may comprise magnetic inductive or short-range RF telemetry schemes as already described. Should the clinician programmer 90 lack a communication antenna, a communication head or wand 94 may be wired to the computer which has a communication antenna. Thus, the ETS 70 and the clinician's programmer 90 and/or its communication head 94 may include antennas compliant with the telemetry scheme chosen. Clinician programmer 90 may be as described in U.S. Patent Application Publication 2015/0360038. External controller 50 (FIG. 2) may also communicate with the ETS 70 to allow the patient means for providing or adjusting the ETS 70's stimulation program.

At the end of the trial stimulation phase, a decision is made whether to abandon stimulation therapy, or whether to provide the patient with a permanent IPG 10 such as that shown in FIGS. 1A and 1B. Should it be determined that stimulation therapy is not working for the patient, the leads 14 or 15 can be explanted from the patient's spinal column 60 and incision 62 closed in a further surgical procedure.

By contrast, if stimulation therapy is effective, IPG 10 can be permanently implanted in the patient as discussed above. (“Permanent” in this context generally refers to the useful life of the IPG 10, which may be from a few years to a few decades, at which time the IPG 10 would need to be explanted and a new IPG 10 implanted). Thus, the IPG 10 would be implanted in the correct location (e.g., the buttocks) and connected to the leads 14 or 15, and then temporary incision 62 can be closed and the ETS 70 dispensed with. The result is fully-implanted stimulation therapy solution. If a particular stimulation program(s) had been determined during the trial stimulation phase, it/they can then be programmed into the IPG 10, and thereafter modified wirelessly, using either the external programmer 50 or the clinician programmer 90.

### SUMMARY

Aspects of the disclosure provide a neuromodulation system comprising: an external device comprising a graphical user interface (GUI) for programming an implantable stimulator device, wherein the implantable stimulator device comprises a plurality of thermodes configured to contact a patient's tissue, wherein the external device comprises a control circuitry programmed to execute at least a thermopole algorithm, wherein the thermopole algorithm is configured to: receive, via the GUI of the external device, one or more inputs indicating one or more prescribed thermopoles in the patient's tissue, and based on the received one or more inputs, provide the thermal stimulation parameters to the implantable stimulator device for generating the one or more prescribed thermopoles. According to some embodiments, the control circuitry is further programmed to execute at least a bioheat model, wherein the bioheat model is configured to model a thermal response of the patient's tissue to thermal stimulation provided to the patient's tissue by the one or more of the plurality of thermodes and select one or more thermal stimulation parameters for providing the one or more prescribed thermopoles. According to some embodiments, the GUI comprises a representation of the one or more thermodes in relation to the patient's tissue and is configured to represent the one or more prescribed thermopoles. According to some embodiments, the bioheat model comprises a finite element model (FEM) comprising modeled tissue comprising one or more of vertebrae, surrounding soft-tissues, epidural fat, meninges, cerebrospinal fluid, or spinal cord. According to some embodiments, selecting the one or more thermal stimulation parameters for providing the one or more prescribed thermopoles comprises: determining desired thermal values at a plurality of spatial points within the patient's tissue, selecting a plurality of constituent thermal sources adjacent one or more thermodes of the plurality of thermodes, determining relative strengths of the constituent thermal sources that, when combined, result in estimated thermal values at the spatial points that best matches the desired thermal values at the spatial points, and selecting a percentage of thermal power to be associated with each of the thermodes based on the determined strengths of the constituent thermal sources. According to some embodiments, selecting the one or more thermal stimulation parameters for providing the one or more prescribed thermopoles further comprises: estimating thermal parameter values per unit power generated by each of the constituent thermal sources at the plurality of spatial points, and generating an m×n transfer matrix from the estimated thermal parameter values per unit power, where m equals the number of spatial points and n equals the number of constituent thermal sources, and wherein the relative strengths of the constituent thermal sources are determined using an optimization function that includes the transfer matrix and the desired thermal parameter values. According to some embodiments, the optimization function is |φ−Aj|2, where φ is a m-element vector of the desired thermal parameter values, A is the transfer matrix, and j is an n-element vector of the strengths of the constituent current sources. According to some embodiments, the GUI comprises a search mode configured to program the implantable stimulator device to elicit electrical stimulation causing paresthesia. According to some embodiments, the GUI comprises a horizontal view and a coronal view. According to some embodiments, the one or more thermodes comprise one or more thermal elements selected from the group consisting of IR LEDs, low powered lasers, ultrasonic heating elements, piezoelectric heating elements, radio frequency heating elements, and resistive heating elements. According to some embodiments, the one or more thermodes comprise electrodes configured to impart joule heating to the patient's tissue. According to some embodiments, the one or more thermodes comprise electrodes configured to impart joule heating to the patient's tissue and wherein the bioheat model models the thermal response of the patient's tissue to thermal stimulation based on RMS intensity of joule heating imparted at the one or more electrodes. According to some embodiments, the bioheat model models the thermal response of the patient's tissue to thermal stimulation based on a power law function of the RMS intensity corresponding to the formula ΔT=A×RMSβ, where ΔT is differences in temperature corresponding to different waveforms, β is a power, and A is a proportionality constant. According to some embodiments, β is a value of 1.4 to 3.5. According to some embodiments, the GUI provides a selection for setting a time course of thermal stimulation and wherein the thermopole algorithm derives thermal stimulation parameters for providing an RMS value as a function of time configured to maintain the time course of thermal stimulation. According to some embodiments, the thermal stimulation parameters for providing an RMS value as a function of time comprise one or more burst patterns stimulation. According to some embodiments, the thermal stimulation parameters for providing an RMS as a function of time comprise one or more continuous charge-balanced waveforms configured to maintain time-varying RMS. According to some embodiments, the external device is configured to receive one or more signals from one or more temperature sensors of the implantable stimulation device. According to some embodiments, the external device is configured to receive one or more signals from one or more temperature sensors of the implantable stimulation device and wherein the bioheat model is modified based on the one or more signals from the one or more temperature sensors. According to some embodiments, the GUI is configured to represent a temperature map of the patient's tissue based on the one or more signals from the one or more temperature sensors.

Further aspects of the disclosure provide an implantable stimulator device, comprising: one or more leads configured for implantation in a patient, the one or more leads comprising a plurality of thermodes, and a control circuitry programmed to: cause one or more of the plurality of thermodes to issue thermal stimulation to the patient's tissue, wherein the thermal stimulation is calculated, based on a thermopole algorithm, to elicit a thermopole in the patient's tissue. According to some embodiments, the one or more thermodes comprise one or more thermal elements selected from the group consisting of IR LEDs, low powered lasers, ultrasonic heating elements, piezoelectric heating elements, radio frequency heating elements, and resistive heating elements. According to some embodiments, the one or more thermodes comprise a plurality of electrodes configured to impart joule heating to the patient's tissue. According to some embodiments, the electrodes of the plurality of electrodes have an inter-electrode distance of less than 1 mm. According to some embodiments, the electrodes of the plurality of electrodes have an inter-electrode distance of less than 0.5 mm. According to some embodiments, the leads further comprise one or more temperature sensors. According to some embodiments, the electrodes have an area of less than 1 cm².

Further aspects of the disclosure provide method of providing thermal stimulation to a patient's tissue using an implantable stimulator device comprising one or more leads comprising a plurality of thermodes implanted in the patient, the method comprising: determining one or more desired thermopoles within a target tissue, using a thermopole algorithm, determining thermal stimulation parameters for two or more of the plurality of thermodes, and applying thermal stimulation at the one or more of the plurality of thermodes using the determined thermal stimulation parameters. According to some embodiments, the one or more desired thermopoles are determined based at least on a bioheat model. According to some embodiments, the bioheat model comprises a finite element model (FEM) comprising modeled tissue comprising one or more of vertebrae, surrounding soft-tissues, epidural fat, meninges, cerebrospinal fluid, or spinal cord. According to some embodiments, the target tissue is a spinal cord, dorsal root ganglion, or one or more dorsal roots and wherein the one or more leads are implanted in epidural fat. According to some embodiments, two or more of the plurality of thermodes are 2 mm to 6 mm distant from the target tissue. According to some embodiments, two or more of the plurality of thermodes have an inter-thermode distance of 0.8 to 2.5 times the distance of either of the thermodes to the target tissue. According to some embodiments, two or more of the plurality of thermodes have an inter-thermode distance of less than 1 mm. According to some embodiments, two or more of the plurality of thermodes have an inter-thermode distance is than the distance from either thermode to the target tissue. According to some embodiments, the thermal stimulation causes a temperature increase of at least 0.5° C. in the target tissue. According to some embodiments, the thermal stimulation causes a temperature increase of at least 0.5° C. to 4.0° C. in the target tissue. According to some embodiments, the thermopole is maintained for greater than 10 minutes. According to some embodiments, the method further comprises providing electrical neuromodulation in addition to thermal stimulation.

Further aspects of the disclosure provide a non-transitory computer readable media comprising instructions executable on an external device comprising a graphical user interface (GUI) for programming an implantable stimulator device, wherein the implantable stimulator device comprises a plurality of thermodes configured to contact a patient's tissue, wherein the instructions a thermopole algorithm, wherein the thermopole algorithm, when executed, is configured to: receive, via the GUI of the external device, one or more inputs indicating one or more prescribed thermopoles in the patient's tissue, select one or more thermal stimulation parameters for providing the one or more prescribed thermopoles, and provide the thermal stimulation parameters to the implantable stimulator device for generating the one or more prescribed thermopoles. According to some embodiments, the non-transitory computer readable media further comprises a bioheat model, wherein the bioheat model, when executed, is configured to model a thermal response of the patient's tissue to thermal stimulation provided to the patient's tissue by the one or more of the plurality of thermodes. According to some embodiments, the non-transitory computer readable media further comprises instructions for any of the concepts described above.

### BRIEF DESCRIPTION OF THE DRAWINGS

FIGS. 1A and 1B respectively show an Implantable Pulse Generator (IPG) in plan and cross-sectional views, in accordance with the prior art.

FIG. 2 shows a hand-held external controller for communicating with an IPG, in accordance with the prior art.

FIG. 3 shows a clinician programming system for communicating with an IPG or an External Trial Stimulator (ETS), in accordance with the prior art.

FIGS. 4A and 4B show aspects of the spinal cord and related neural anatomy

FIGS. 5A and 5B show a stimulation program.

FIGS. 6A and 6B show SGC and DH initiated pathways of MoAs of thermal stimulation, respectively.

FIG. 7 shows aspects of MoAs of thermal stimulation.

FIG. 8 shows aspects of a system for providing thermal stimulation.

FIG. 9 shows a lead and circuitry for an implantable stimulator device.

FIGS. 10A and 10B show aspects of stimulation circuitry and stimulation using biphasic pulses.

FIG. 11 shows aspects of thermopole generation using two thermodes.

FIG. 12 shows an example of the inputs and outputs of an embodiment of a thermopole algorithm.

FIG. 13 shows an array of thermal field sample positions and an array of thermodes, as used in embodiments of a thermopole algorithm.

FIGS. 14A-14C show matrices used according to embodiments of a thermopole algorithm.

FIG. 15 shows an example workflow for delivering dosed and calibrated thermal stimulation.

FIG. 16 shows aspects of a graphical user interface (GUI).

FIG. 17 shows aspects of a GUI.

FIG. 18 shows aspects of a GUI.

FIG. 19 shows aspects of a GUI.

FIG. 20 shows a time course of RMS power for a pattern of waveform envelopes.

FIG. 21 shows a continuous, constantly fluctuating waveform, customized to hold a time-varying RMS for temperature control.

FIG. 22 shows a table showing temperature increases predicted under varied stimulation parameters.

FIG. 23 shows the order of simulated tissues and predicted maximum temperature increases at locations corresponding to Lead position (“Lead”), Spinal Cord surface (“SC”), and dorsal Root surface (“Root”) for both passive heating and active heating conditions.

### DESCRIPTION

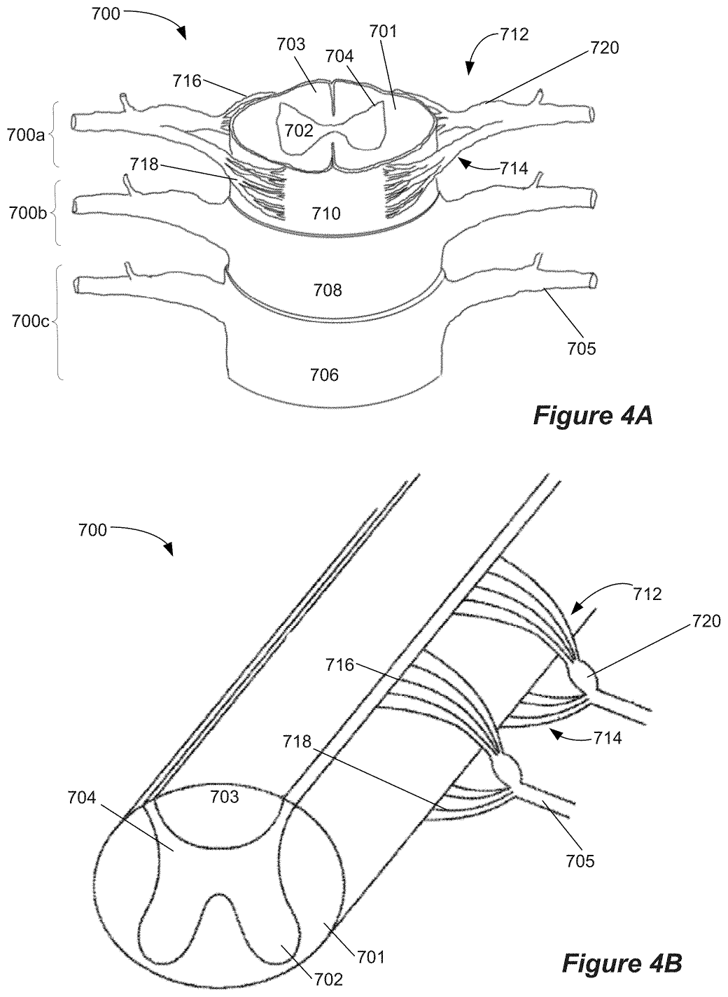

Various embodiments described herein involve neural stimulation and thermal stimulation of a patient's neural tissue. Examples include spinal cord modulation, i.e., spinal cord stimulation (SCS) as well as stimulation and sensing of related neural anatomy. Additional embodiments may include deep brain stimulation (DBS), peripheral nerve stimulation (PNS), and the like. Focusing on SCS, a brief description of the anatomy and physiology of the spinal cord is provided herein to assist the reader. FIGS. 4A and 4B illustrate, by way of example, a portion of a spinal cord 700 including white matter 701 and gray matter 702 of the spinal cord. A typical transverse section of the spinal cord includes a central “butterfly” shaped central area of gray matter 702 substantially surrounded by an ellipse-shaped outer area of white matter 701. The white matter of the dorsal column (DC) 703 includes mostly large myelinated axons that form afferent fibers that run in an axial direction. The dorsal portions of the “butterfly” shaped central area of gray matter are referred to as dorsal horns (DH) 704. In contrast to the DC fibers that run in an axial direction, DH fibers can be oriented in many directions, including laterally with respect to the longitudinal axis of the spinal cord. The gray matter 702 includes cell bodies, synapse, dendrites, and axon terminals.

Referring to FIG. 4A, the spinal cord is enclosed within three layers of tissue, collectively called the meninges. The outer layer of the meninges, called the dura mater 706, is shown in spinal cord segment 700c. The dura mater has been removed in spinal cord segment 700b to reveal the middle meninges, called the arachnoid 708. The innermost meninges, the pia mater 710, is shown in spinal cord segment 700a.

Examples of spinal nerves 705 are also illustrated. Upon removal of the meningeal layers, it is seen that each spinal nerve 705 splits into a dorsal root (DR) 712 and a ventral root 714, each of which comprise subdivisions referred to as rootlets. In FIG. 4A, the dorsal rootlets are labeled 716 and the ventral rootlets are labeled 718. The dorsal root also includes a structure called the dorsal root ganglion (DRG) 720, which comprises cell bodies of the afferent neurons. The dorsal root 712 contains afferent neurons, meaning that they carry sensory signals into the spinal cord, and the ventral root 714 functions as an efferent motor root. The dorsal and ventral roots join to form mixed spinal nerves 705.

While the full mechanisms of pain relief using SCS is not completely understood, it is believed that the perception of pain signals is inhibited via the gate control theory of pain, which suggests that enhanced activity of innocuous touch or pressure afferents via electrical stimulation creates interneuronal activity within the DH 704 of the spinal cord that releases inhibitory neurotransmitters (Gamma-Aminobutyric Acid (GABA), glycine), which in turn, reduces the hypersensitivity of wide dynamic range (WDR) sensory neurons to noxious afferent input of pain signals traveling from the dorsal root (DR) neural fibers that innervate the pain region of the patient, as well as treating general WDR ectopy. Consequently, the large sensory afferents of the DC nerve fibers have been targeted for stimulation at an amplitude that provides pain relief.

An example of stimulation pulses as prescribed by an example stimulation program and as executable by the IPG or ETS 70 is illustrated in FIGS. 5A and 5B. As shown in FIG. 5A, electrode E4 is selected as the anode and electrode E5 is selected as the cathode. FIG. 5B illustrates the waveforms of the stimulation pulses delivered by E4 and E5. In the example shown, each stimulation pulse is biphasic, meaning it comprises a first pulse phase followed essentially immediately thereafter by an opposite polarity pulse phase. The pulse width (PW) could comprise the duration of either of the pulse phases individually as shown, or could comprise the entire duration of the biphasic pulse including both pulse phases. The frequency (f) and amplitude (A) of the pulses is also shown. Although not shown, monophasic pulses—having only a first pulse phase but not followed by an active-charge recovery second pulse phase—can also be used. The pulses as shown comprise pulses of constant current, and notice that the amplitude of the current at any point in time is equal but opposite such that current injected into the patient's tissue by one electrode (e.g., E4) is removed from the tissue by the other electrode (E5). Notice also that the area of the first and second pulses phases are equal, ensuring active charge recovery of the same amount of charge during each pulse phase. Although not shown, more than two electrodes can be active at any given time. For example, electrode E4 could comprise an anode providing a +10 mA current pulse amplitude, while electrodes E3 and E5 could both comprise cathodes with −7 mA and −3 mA current pulse amplitudes respectively. Biphasic pulses are particularly beneficial when pulses are issued at higher frequencies, although they may be used at lower frequencies as well.

The inventors have discovered that targeted application of thermal stimulation instead of, or in addition to, electrical stimulation to a patient's neural elements facilitates pain relief and/or other therapeutic benefits. Thus, aspects of this disclosure provide methods and systems for delivering and controlling dosed and calibrated thermal stimulation to specific target tissues using an implantable stimulator device. For example, the methods and systems described herein may be used to thermally stimulate specific regions of the epidural space and/or spinal cord and/or DRG/SGC and/or spinal verve roots for producing a neuroprotective and/or anti-inflammatory effect via the forced expression of heat shock proteins through mild heating, as discussed in more detail below.

Aspects of the disclosure provide:
(1) bioheat models based on physiological and thermal properties of target anatomy, which allows dosed and calibrated thermal stimulation to be delivered to the target anatomy. The bioheat model predicts the thermal fields, referred to herein as “thermopoles,” that arise in the target tissue as a result of thermal stimulation. A derivation of an embodiment of a bioheat model is detailed in the Examples below.
(2) implantable stimulator devices configured to provide dosed and calibrated thermal stimulation. The implantable stimulator devices may include one or more thermodes. The term “thermode,” as used herein, refers to an element that acts as a heat source. As described further below, thermodes may be one or more electrodes that provide thermal stimulation via joule heating. Thermodes may also comprise temperature elements that provide thermal stimulation via other heating modalities. The implantable stimulator device may include multiple thermodes and may include multiple types of thermodes. The implantable stimulator device may also include one or more temperature sensors. The implantable stimulator may include control circuitry for controlling the operation of the stimulator, including controlling the delivery of thermal stimulation and/or electrical stimulation, and may also be configured for closed loop feedback (for example, based on temperature measurements) to automatically preserve temperature near the thermodes within a range and automatically adjust different stimulation settings to preserve temperature within that range. The implantable stimulator device may also include one or more electrodes configured to provide electrical stimulation/modulation of neural targets.
(3) algorithms that interact with the bioheat model for deriving thermal stimulation parameters for providing dosed and calibrated thermal stimulation. Such algorithms are referred to herein as “thermopole algorithms.” The thermopole algorithms derive appropriate spatiotemporal thermal output, specifically power output, to elicit desired thermopoles in the target tissue. Aspects of the thermopole algorithms are executed in, and control, the control circuitry (e.g., microcontroller) of the implantable stimulator device. The controller may be constrained in various ways such as charge balance, minimizing total power (while still maintaining a target temperature range), core temperature, surface temperature (skin or environment), heart rate, time of day, patient input, activity (exercise increasing temperature). Thermopoles may be cumulative on core temperature and therefore the controller can consider core temperature or other markers that influence core temperature, via the thermopole algorithm. The target temperature increase can be expressed in absolute terms such that the delta is a differential from the measured or assumed core temperature. As examples, according to some embodiments, if activity (e.g. accelerator, heat rate, breathing rate data) exceeds a threshold the stimulator can be deactivated (or substantially reduced in power) for a set period of time and/or until the activity decreases below the threshold. The thermal stimulation methods and controls may include a safety element and a control element. The total energy dose may be regulated over a user-specific time period. According to some embodiments, the dose/time relationship may involve a time-course of stimulation for controlling the amount of thermal heat induction.
(4) interfaces, such as one or more graphical user interface(s) (GUIs) for configuring and targeting heat delivery into specific targets.

Before discussing the details of the methods and systems disclosed herein, exemplary mechanisms of action (MOAs) provided by thermal stimulation is briefly discussed. Without being bound by theory, thermal stimulation promotes the expression of “heat shock proteins,” which results in reduction of pain sensitization. Heat shock proteins (HSP) are molecular “chaperones” that facilitate protein synthesis and prevent the degradation of gene expression products during thermal stress. Of note, heat shock proteins can be expressed during febrile conditions that involve a temperature rise of as little as 2-3° C. For example, elevated expression of the heat shock protein Hsp70 has been shown to inhibit activation of the pro-neuroinflammatory transcription factor NF-κB. NF-κB is an inflammatory transcription factor that influences the expression of many inflammatory markers in the central nervous system (CNS) and is linked to neuropathic pain. It is known that knocking out NF-κB dependent satellite ganglion cell (SGC) activation reduces expression of neuronal colony stimulating factor 1 (Csf1), which is expressed by neurons. Reduction in Csf1 reduces dorsal horn microglia activation, a hallmark of pain sensitization.

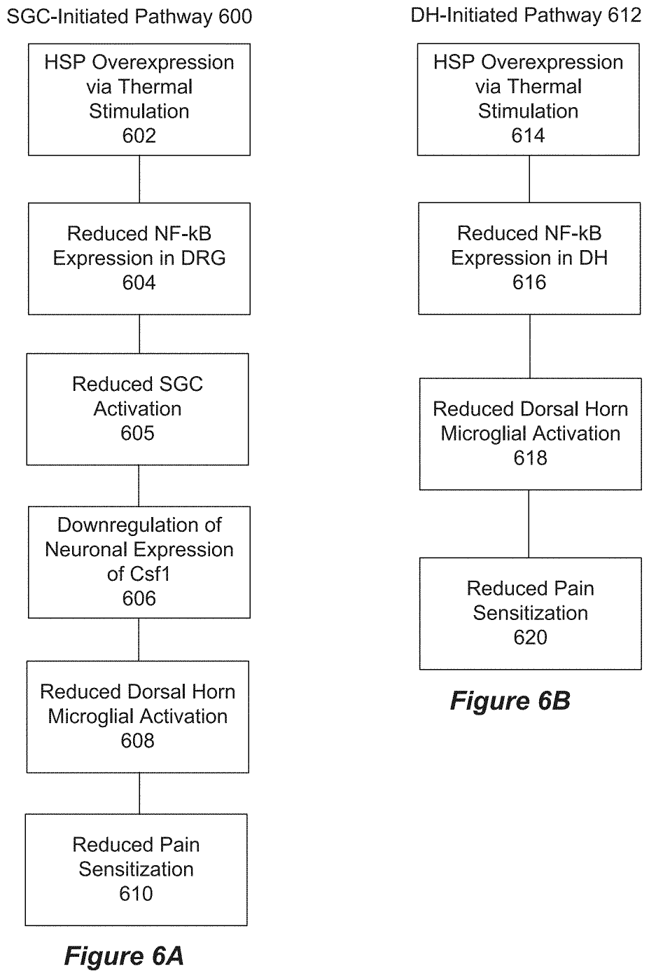

The inventors have invented systems and methods for delivering and controlling dosed and calibrated thermal stimulation to specific regions of the epidural space, spinal cord, DRG, SGC, and/or spinal nerve roots, thereby providing a neuroprotective and/or anti-inflammatory effect via the expression of heat shock proteins through mild heating. The systems and methods described herein can elicit reduction of pain sensitization through one or both of pathways illustrated in FIGS. 6A and 6B.

FIG. 6A illustrates an SGS-initiated pathway 600. According to pathway 600 thermal stimulation of neural elements, for example within the DC, stimulates HSP overexpression 602. Elevated HSP reduces NF-κB in the DRG 604, which results in reduced SGC activation 605. Reduced SGC activation results in downregulation of neuronal expression of Csf1 606, which results in reduced dorsal horn microglial activation 608, resulting in reduced pain sensitization 610.

FIG. 6B illustrates a DH-initiated pathway 612. According to pathway 612 thermal stimulation of neural elements, for example within the DC, stimulates HSP overexpression 614. Elevated HSP reduces NF-κB in the DH 616, which results in reduced dorsal horn microglial activation 618, resulting in reduced pain sensitization 620.

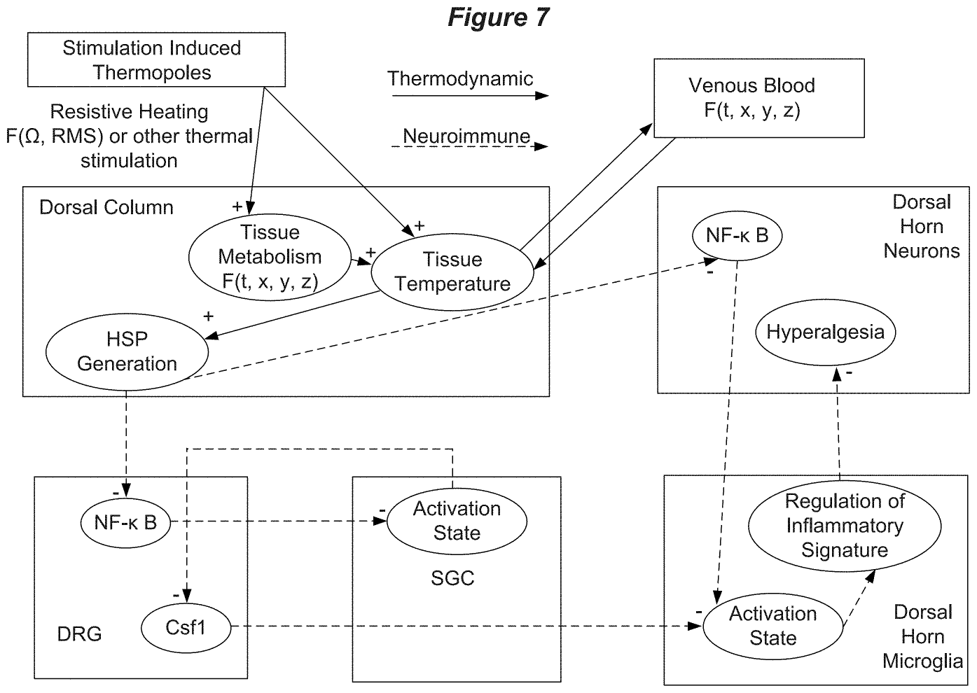

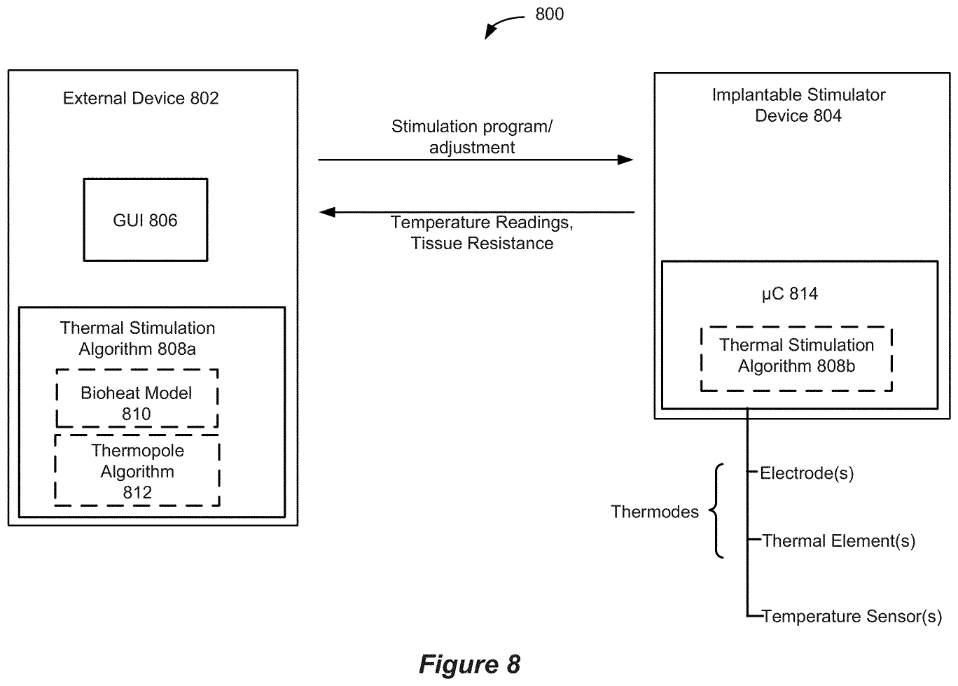

FIG. 7 illustrates aspects of the pathways described above in relation to the relevant neural anatomy. In FIG. 7, thermodynamic interactions are shown with solid arrows and neuroimmune/secretory interactions are shown with dashed arrows. Thermal stimulation induces one or more temperature fields, referred to herein as “thermopoles” in the tissue. Thermopoles will be discussed in more details below. In the case of electrical stimulation (e.g., SCS), resistive heating of the tissue (e.g., tissue of the epidural space, dorsal column, etc.) is a function of the resistance of the tissue and the RMS power dissipated within the tissue. Thermal stimulation within the epidural space heats dorsal column tissue via heat conduction. The heating of dorsal column tissue is time and location dependent and can be predicted based on the bioheat model. As the temperature of the dorsal column tissue rises the metabolism rate of the tissue can increase, further increasing the tissue temperature. Increasing the tissue temperature can impact venous blood flow within the tissue. Increased temperature within the dorsal column also stimulates increased expression of HSP. Each of these factors can be included in the bioheat model.

Increased HSP expression within the dorsal column reduces NF-κB expression in the DRG and/or in the DH. Reduced NF-κB expression in the DRG can downregulate SGC activation, thereby downregulating Csf1 expression in the DRG resulting in reduced dorsal horn microglial activation. Reduced NF-κB expression in the DH neurons can also result in reduced dorsal horn microglial activation. Reduced dorsal horn microglial activation impacts regulation of inflammatory signatures in the dorsal horn microglia, which reduces hyperalgesia of the dorsal horn neurons (i.e., reduces pain sensitization).

According to some embodiments, thermal energy can be provided to a target tissue via joule heating associated with electrical stimulation, such as electrical stimulation provided in traditional or high frequency neuromodulation. Particularly, the emergence of kilohertz frequency (1-10 KHz) spinal cord stimulation (kHz-SCS) for the treatment of neuropathic pain implicates new mechanisms of actions (MoA). Divergent clinical observations for conventional rate SCS and kHZ-SCS suggest difference in MoA which in turn inform distinct programming optimization strategies. Notably, kHZ-SCS can provide an analgesic and side-effects profile distinct from conventional frequency (˜100 Hz) SCS and undermine traditional models of SCS mechanism, such as those mentioned above. For example, kHz-SCS does not produce the paresthesias associated with dorsal column activation in conventional SCS, and recent studies seemingly rule out direct activation of dorsal column fibers as the primary mechanism of action of kHz-SCS pain relief. The wash-in times associated with kHz-SCS treatment tend to be longer than those associated with conventional rate SCS. Further indicating distinct MoA, kHz-SCS waveforms involve simultaneous decrease in pulse duration (well below membrane time constants) and increase in pulse frequency (beyond axon refractory periods) that challenge conventional models of stimulation. Clinical responses specifically related to unpleasant sensations generated by higher amplitudes of kHz stimulation further reveal the deficiency of supra-perception amplitude kHz-SCS.

Since the decrease in interpulse-interval (e.g. from 10 ms at 0.1 KHz to 0.1 ms at 10 KHz) is more drastic than the decrease in pulse duration (e.g. from 100 μS per phase at 0.1 KHz to 40 μS per phase at 10 KHz), kHZ stimulation is associated with higher duty cycle. The RMS power of a rectangular waveform varies positively with the square root of its duty cycle. Through the principle of joule heating, the power of current flow from an implanted lead can produce temperature increases around the lead. Thus, kHz stimulation deposits more power in the tissue than conventional spinal cord stimulation and is therefore more likely to significantly heat the tissue immediately surrounding the stimulation site. A temperature increase and resultant thermal conduction into the spinal cord can, in turn, affect neuronal function (e.g., via alteration of ion channel or neurotransmitters dynamics) and related biological functions (e.g., via vasodilation, heat shock protein expression) depending on the degree of change. Tissue heating further encourages the expression of anti-inflammatory agents, such as heat shock proteins, over a period of time consistent with the extended wash-in times of kHz-SCS treatment.

Any form of electrical stimulation produces passive heating and the extent of induced temperature increases are specific to both the stimulation and local tissue properties, and many stimulation and environmental parameters may affect the degree to which heating occurs. Key stimulation parameters are the stimulation waveform (based on stimulator programming) and electrode montage (based on lead placement), which together with tissue anatomy and electrical conductivity determine joule heat deposition. An implanted stimulator may be a constant energy source which will produce unlimited temperature increases without passive (e.g. heat conduction by CSF) or active (e.g. spinal tissue blood perfusion) heat dissipation by the tissue. As such, heating analysis depends on tissue properties such as thermal conductivity, metabolic rate, and blood perfusion; not only of the stimulation target but also of the surrounding tissues. The local environment around SCS leads is especially conducive to temperature increases, namely the low conductivity of fat and enclosed anatomy of the vertebral canal. Taken together, if heating due to these factors is sufficient during kHz frequency neuromodulation to produce the previously described beneficial responses, then joule heating by SCS can be an adjuvant mechanism underlying therapy. The inventors have determined that an increased duty-cycle (and so power) of High-Rate spinal cord stimulation produces significant temperature increases in the spinal cord.

Aspects of the disclosure relate to systems for providing dosed and calibrated thermal stimulation to specific target tissues within a patient. FIG. 8 schematically illustrates components of such a system 800. Each of the components will be described in more detail below.

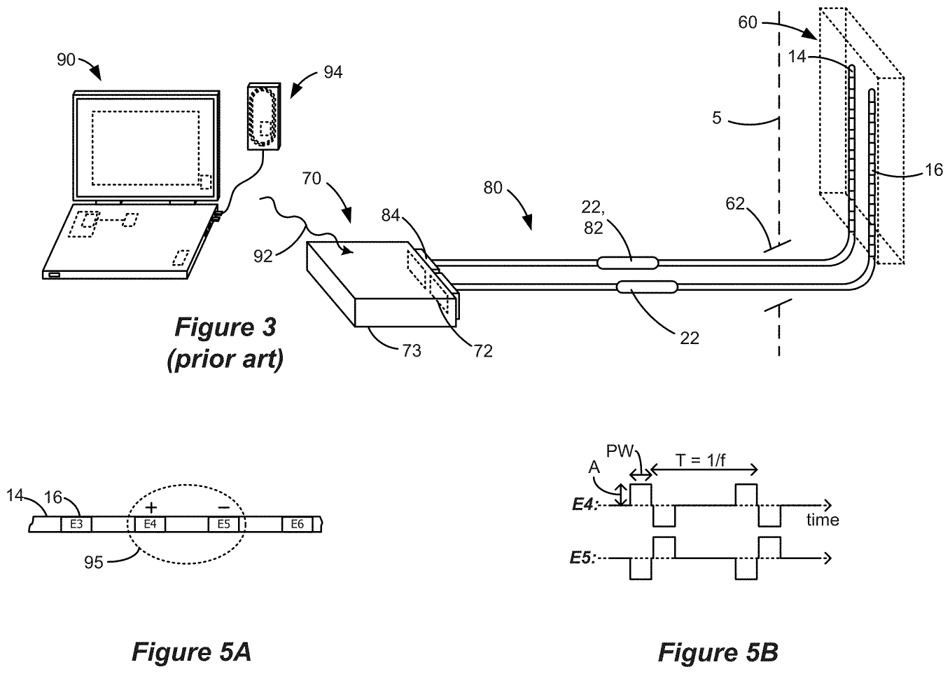

The system 800 can include an external device 802, which can be generally any specifically programmed computing device. Examples of external computing devices include devices such as a clinician programmer 90 or external controller 50 described above with reference to FIGS. 2 and 3, which can be used to interact with the implantable stimulation device. An example of a system for interacting with an implantable stimulation device is described in “Precision Spectra™ System Programming Manual,” Boston Scientific Corp., 90834018-18 Rev A (2016). Other examples of suitable external devices include appropriately programmed computing devices, such as tablets or the like, executing appropriately programmed applications. The external device 802 can be configured to transmit data, for example stimulation parameters to the implantable stimulation device 804 and to receive data, such as temperature readings, resistance measurements, etc., from implantable stimulation device. One skilled in the art will understand that the external device 802 will comprise instructions that can be stored on non-transitory machine-readable media, such as magnetic, optical, or solid-state memories. Such memories may be within the external device 802 itself (i.e., stored in association with control circuitry, storage medium (magnetic, optical, etc.)), or readable by the system (e.g., memory sticks or disks). Such memories may also include those within Internet or other network servers, such as an implantable medical device manufacturer's server or an app store server, which may be downloaded to the external system.

Using the external device 802, the user can presented with a user interface, such as a graphical user interface (GUI) 806, which is configured to present the user with a representation of the electrical signals, thermal stimulation parameters and/or temperature readings sensed at the various available implanted electrodes, thermodes and/or temperature sensors, with buttons that allow the user to manually change the stimulation intensity or other stimulation parameter in the implantable stimulation device 804. Aspects of the GUI 806 and how a user can interact with the GUI are discussed in more detail below.

The external device 802 can be configured with aspects of a thermal stimulation algorithm 808a. It should be noted here that some aspects of the thermal stimulation algorithm may be embodied within the external device 802 and some aspects may be embodied within the implantable stimulator 804. The thermal stimulation algorithm 808a may be conceptually thought of as comprising two aspects: a bioheat model 810 and a thermopole algorithm 812. While those two aspects are illustrated separately in FIG. 8, it should be appreciated that there may not be a clear distinction between the two aspects as they may be programmed and may interact as a single logical component.

Embodiments of the bioheat model 810 provide models, such as finite-element models (FEMs), for predicting the degree of tissue temperature rises driven by SCS joule heating as well as other heating modalities, as described below. The Examples describe an embodiment of a FEM model wherein a human spinal cord is simulated as a computer-aided design (CAD)-derived model comprising seven compartments namely vertebrae (e.g., lower thoracic region, T8-T11), intervertebral disc, surrounding soft-tissues (minimally perfused), epidural fat, meninges, cerebrospinal fluid, and spinal cord (white matter and grey matter combined) and solved using the applicable tissue density, specific heat, temperature, electrical conductivity, and thermal conductivity of each of the compartments. Greater or fewer compartments may be included in the simulation.

As described in the Examples, heat delivery is primarily a function of power imparted into the tissue, such as RMS intensity (e.g., RMS power), in the case of electrical stimulation/modulation. Aspects of the thermopole algorithm 812 interacts with the bioheat model 810 to predict the temperature field, i.e., the thermopole(s), arising in the tissue based on given thermal stimulation waveform parameters, time course of stimulation, thermode placement and geometries, and the like. In the case of electrical stimulation, the power transmitted due to current flow is equal to the (RMS current) x resistance. The RMS of the stimulation waveform is tied to the amplitude and waveform shape. As pertains to thermal stimulation, generally any waveform shape can be used and the amplitude, pulse width, duty cycle and pulse rate (frequency) can be controlled to modulate the power delivered. The thermopole algorithm 812 can be used to predict the thermal response of the modeled tissue to stimulation having a particular set of parameters and, moreover, can be used to derive stimulation parameters for obtaining a particular desired thermal stimulation objective.

Another aspect of the thermopole algorithm 812 can be used to steer and focus thermopoles in the target tissue given a selection of available thermodes. Such aspects of the thermopole algorithm may be thought of as thermal analogues to techniques for steering electric field potentials, i.e., “target poles,” described in U.S. Pat. No. 8,412,345, issued Apr. 2, 2013 (the entire contents of which are hereby incorporated by reference) and in U.S. Provisional Patent Application No. 62/598,114, filed Dec. 13, 2017 (the contents of which are hereby incorporated by reference). Thermopole steering is discussed in more detail below.

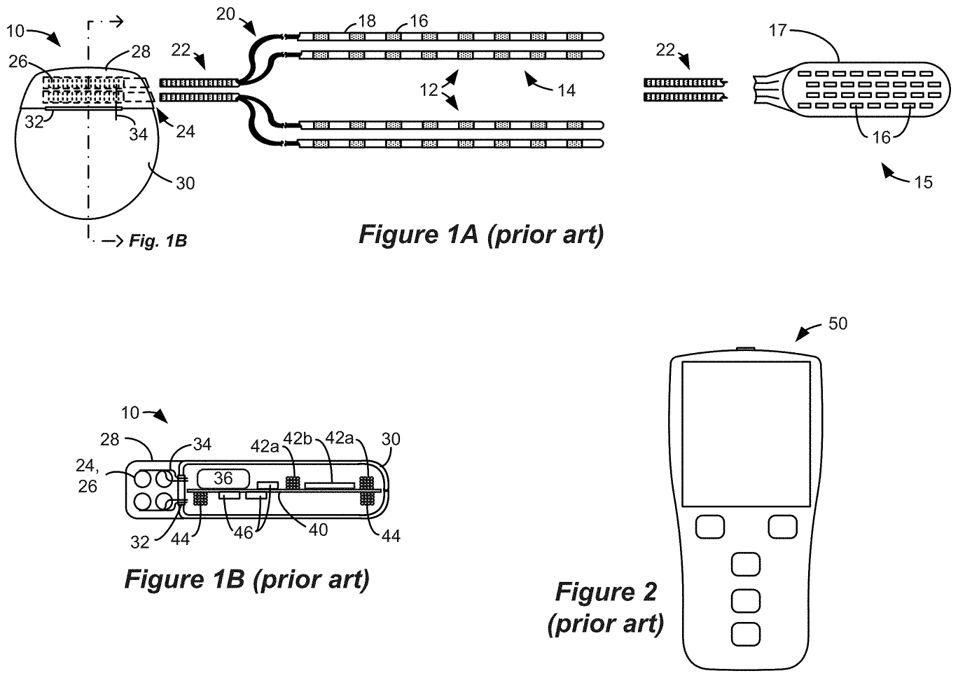

System 800 includes an implantable stimulator device 804. Examples of an implantable stimulator device include improved IPGs and ETSs as described above with reference to FIGS. 1-3. Note that, for simplicity, ETSs are referred to herein as an example of an implantable stimulator device, even though, by definition, they are not implanted within a patient during the trial phase. However, they may include any of the functionality ascribed to an IPG or other implantable stimulator device and are therefore included as an example of an implantable stimulator device for the purposes of this discussion.

The implantable stimulator device 804 includes a microcontroller 814 that may embody one or more aspects of the thermal stimulation algorithm 808b (including aspects of the bioheat model and/or the thermopole algorithm). As mentioned above, some aspects of the thermal stimulation algorithm may be executed/performed in the external device while other aspects are executed/performed in the implantable stimulator device 804. The implantable stimulator device may include one or more leads 14, which include one or more thermodes, such as electrodes and/or thermal elements and may include one or more temperature sensors. Further aspects of the implantable stimulator device are discussed below.

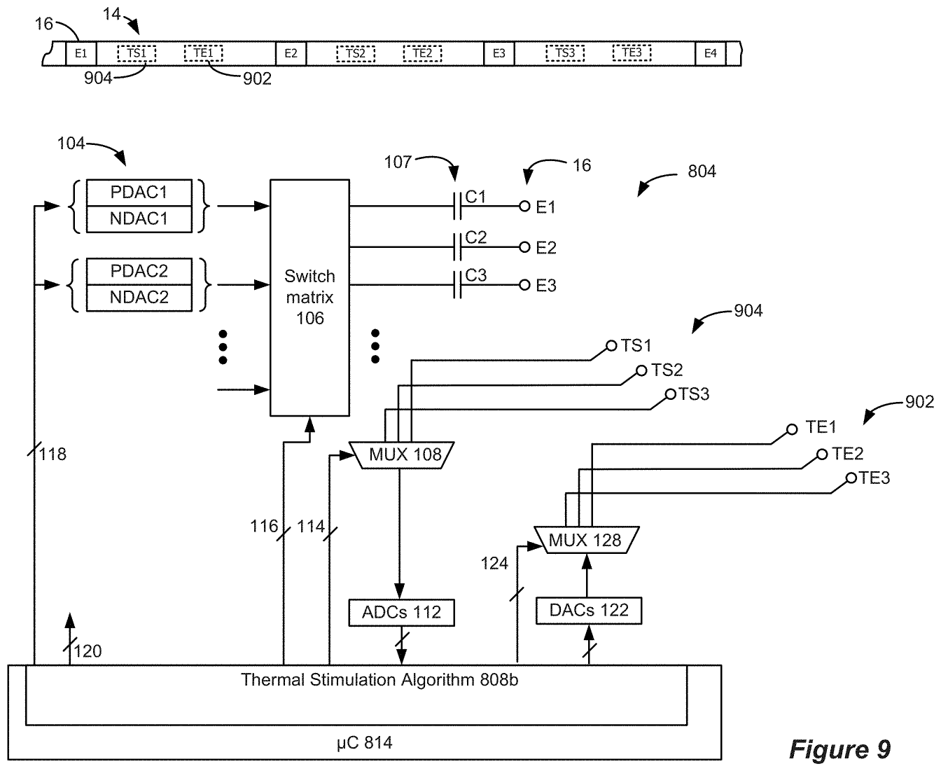

FIG. 9 shows a lead 14 and circuitry for an implantable stimulator device 804. The illustrated lead 14 includes a plurality of electrodes E1, E2, E3, E4, . . . , (collectively 16), a plurality of thermal elements TE1,TE2, TE3, . . . , (collectively 902), and a plurality of temperature sensors TS1, TS2, TS3, . . . , (collectively 904). It should be noted that some embodiments may not include all these elements. For example, one embodiment of a lead 14 may include only electrodes. An alternative embodiment may include electrodes and one or more temperature sensors. An alternative embodiment may include only thermal elements or may include thermal elements and one or more temperature sensors. It should also be noted that FIG. 9 illustrates a percutaneous lead 14. However, other types of leads, such as paddle leads, directional leads, etc. can be used.

According to some embodiments, the electrodes 16 may be configured to provide electrical stimulation as is known for electrical-based neuromodulation. The electrodes may also be configured to provide joule heating as described above. Thus, some embodiments may provide both modalities of stimulation/modulation, i.e., both electrical and thermal, using electrodes on the same lead or electrodes on a combination of leads. It should be noted that any given electrode may be configured to provide both thermal and electrical stimulation. For example, a waveform may be prescribed that provides both electrical neuromodulation and prescribed thermal modulation. As used herein, an electrode implemented for providing prescribed thermal stimulation may be referred to as a “thermode.”

According to some embodiments, the shape and size of at least some of the electrodes can be optimized for temperature lead fields and inducement of thermopoles. For example, decreasing the electrode area to 16 mm² and to 8 mm² can provide an exponential increase in temperature rise. Small electrodes inherently allow for more proximal electrode placement. Small inter-electrode distances can minimize direct neuronal polarization while increasing temperature rise. For example, an inter-electrode distance of less than 2 mm or less than 0.5 mm can provide enhanced temperature rise while minimizing direct activation. In this way, less power may be applied to achieve comparable temperature rise. For an inter-electrode distance of less than 2 mm a stimulation RMS of 0.5 to 3 mA may be preferred. Small inter-electrode distances can be accompanied by increases in stimulation frequency. Likewise, the surface composition of one or more of the electrodes may be optimized for thermal delivery, for example, by increasing the roughness of the surface. According to some embodiments, one or more of the electrodes may be covered by a thin resistive layer to provide a joule heat spike at the interface.

According to some embodiments a directional lead can be used and adjacent or proximal electrodes in the same lead segment can be used for temperature increases. For example, in a lead with four electrodes (quarters) per segment, adjacent electrodes can be used for thermal stimulation or electrodes on opposite sides of the lead may be used. Adjacent electrodes may be used to generate a local hot spot of temperature. Opposite electrodes may be used to enhance deeper temperature penetration while still controlling other forms of polarization. The selection of electrodes on a directional lead may be informed by impedance measurements across all electrodes, the bioheat model(s), as well as feedback from sensors. When sensors are used with directional leads, the sensors can be distributed radially around the lead with either one temperature sensors per electrode, in which case the sensors may be painted between electrodes, or one temperature sensors for two electrodes in which case the sensor may be positioned centered on electrode. For adjacent electrodes, the temperature rise primarily located at the junction.

The temperature increase produced by an electrode is a function of the electrode perimeter length and shape. Circular electrodes generate heating proportional diameter with a diameters less than 1 cm or less than 0.5 cm being preferred for application with enhance temperature rise, according to some embodiments.

Referring again to FIG. 9, the lead 14 may include one or more temperature sensors 904. Examples of temperature sensors 904 can include thermocouples or other thermosensitive electrical elements such as thermos-resistors. Such elements can be in the middle of the lead, in the non-conducting elements of the lead, next to a conducting electrode, under and touching an electrode, just outside but touching the lead, or floating in the tissue at some distance from the electrode or lead. Alternatively, the temperature sensor(s) 904 may be optical in nature where light is applied via a local source (e.g. photo-diode) or fiber optic. Alternatively, the light source may be configured remotely, for example, in the can of the implantable stimulator device 804, and light may travel through a light guide in the lead and emerge from the lead, for example near a thermode. The lens and light applied may be configured to obtain temperature measurements from a relevant field of view. In any of the above cases there may be arrays of temperature sensors, that may or may not correspond to electrodes, where information from these sensors can be processed together. The thermal stimulation algorithm may consider a bioheat model of the tissue, lead geometry, electrodes used, and the goal of stimulation. According to one embodiment, at least one sensor is integrated into the lead such that when the lead is implanted the sensor is positioned outside the spinal cord. According to one embodiment the temperature sensors are integrated into the surface of the device case in a manner that reports body temperature. For example, the sensor may be integrated portion of the lead wire proximal to the device case. According to some embodiments, sensors integrated around the in the lead may be every 1-3 mm along the lead and within 2 mm of any used thermode. According to some embodiments, when a pad electrode is used, sensors can comprise a gird of density at least 4×4 mm and preferably 3×3 mm, for example. According to some embodiments, the temperature sensor(s) may provide an accuracy of 0.2° C. or preferably 0.1° C.

Referring again to FIG. 9, the lead 14 can include one or more thermal elements 902 as thermodes configured to impart thermal energy to the tissue. Examples of thermal elements 902 can include optical heating elements, such as IR LEDs, low powered lasers or may include ultrasonic/piezoelectronic elements, radiofrequency elements, resistive heating elements, and the like.

As mentioned above, the implantable stimulator device 804 includes control circuitry, such as microcontroller 814 into which aspects of the thermal stimulation algorithm 808b can be programmed. Control circuitry 814 may comprise a microcontroller for example such as Part Number MSP430, manufactured by Texas Instruments, which is described in data sheets at http://www.ti.com/lsds/ti/microcontroller/16bit_msp430/overview.page? DCMP=MCU_other& HQS=msp430, which is incorporated herein by reference. Other types of control circuitry may be used in lieu of a microcontroller as well, such as microprocessors, FPGAs, DSPs, or combinations of these, etc. Control circuitry 814 may also be formed in whole or in part in one or more Application Specific Integrated Circuits (ASICs), as described in U.S. Patent Application Publication 2012/0095529 and U.S. Pat. Nos. 9,061,140 and 8,768,453, which are incorporated herein by reference.

According to embodiments of the implantable stimulation device 804 a bus 118 provides digital control signals to one or more Digital-to-Analog converters (DACs) 104, which are used to produce currents or voltages of prescribed amplitudes (A) for the stimulation pulses, and with the correct timing (PW, f). As shown, the DACs can include both PDACs which source current to one or more selected anode electrodes, and NDACs which sink current from one or more selected cathode electrodes. In this example, a switch matrix 106 under control of bus 116 is used to route the output of one or more PDACs and one or more NDACs to any of the electrodes, which effectively selects the anode and cathode electrodes. Buses 118 and 116 thus generally set the stimulation program for the electrodes 16 of the implantable stimulation device 804. The illustrated circuitry for producing stimulation pulses and delivering them to the electrodes is merely one example. Other approaches may be found for example in U.S. Pat. Nos. 8,606,362 and 8,620,436, and U.S. Provisional Patent Application Ser. No. 62/393,003, filed Sep. 10, 2016. Note that a switch matrix 106 isn't necessarily required, and instead a PDAC and NDAC can be dedicated to (e.g., wired to) each electrode. Notice that the current paths to the electrodes 16 include the DC-blocking capacitors 107, which provide additional safety by preventing the inadvertent supply of DC current to an electrode and to a patient's tissue.

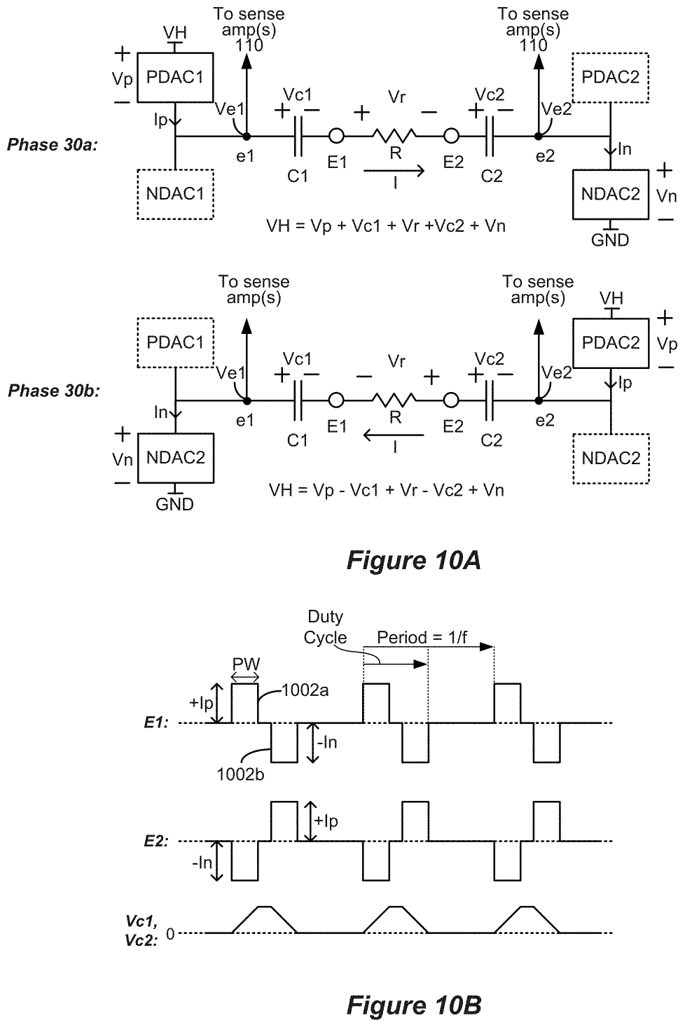

FIGS. 10A and 10B show stimulation occurring using biphasic pulses between electrodes E1 and E2 of FIG. 9. FIG. 10A shows how the stimulation circuitry is biased when producing a current I through the tissue during the first phase 1002a when current I travels from anode electrode E1 to cathode electrode E2, and during the second phase 1002b when current I travels in the opposite direction from anode electrode E2 to cathode electrode E1. The tissue has a resistance R. Note during the first phase 1002a that a selected PDAC1 sources current Ip to electrode node e1 while a selected NDAC2 sinks current In from electrode node e2. During the second phase 1002b, a selected PDAC2 sources current Ip to electrode node e2 and a selected NDAC1 sinks current In from electrode node e1. Ideally, Ip issued from the PDACs equals issued by the NDACs, with both equaling the desired current I. The same PDAC and NDAC could also be used during the two phases if switch matrices are used as part of the design of stimulation circuitry.

FIG. 10B shows various waveforms that are produced when biphasic current pulses are produced at electrodes E1 and E2. Providing a constant current I between the electrodes causes the DC-blocking capacitors C1 and C2 to charge during the first pulse phases 1002a, which causes the voltages across them Vc1 and Vc2 to increase (I=C*dV/dt). Because the second pulse phase 1002b of opposite polarity is charge balanced with the first pulse phase 1002a, Vc1 and Vc2 will decrease during the second pulse phases 1002b and return (ideally) to zero at the end of the second pulse phase 1002b.

As mentioned above, the power dissipated within the tissue (and thus, thermal energy provided to the tissue) is defined by power PW=I²R, where I is the current passed through the tissue and R is the resistance of the tissue. The resistance R of the tissue can be measured by measuring the resistance between the electrode nodes e1 and e2 based on voltages applied at Ve1 and Ve2. Thus, the internal stimulation device 804 can be configured to measure the tissue resistance R. For example, U.S. Pat. No. 9,061,140, issued Jun. 23, 2015 provides examples of measuring tissue resistance using test pulses or therapeutic pulses. The resistance is an aggregate measure across tissue resistance. Measuring resistance across one or more electrode poles, at one of more test frequencies, allows parametrization of the thermal stimulation algorithm 808a/b to guide thermopole stimulation. Resistance may be measured acutely after implant, before each programming phase, or at fixed intervals. Intervals of every 14 days or every 50 days allow for detection and accommodation of tissue lead encapsulations. Impedance measurements may also be impacted by and may inform physiological impacts of thermal stimulation, such as microglia activation.

The resistance R of the tissue can be assumed to be relatively constant over a set programming period. Thus, the power provided to the tissue can generally be controlled by controlling the amplitude of the current provided (e.g., +Ip, of FIG. 10B) and/or the duty cycle of the stimulation. The duty cycle may refer to portion of time during a period which current is flowing. As concerns power dissipation, the polarity of the current is irrelevant. Increasing the duty cycle or increasing the amplitude increases the power provided to the tissue. However, there are other factors such as electrochemical safety, hardware limitations, power consumption, and safety or regulatory compliance that may restrict waveform features. For pulsed stimulation, decreasing the period (1/f) to less than 10 times the pulse width (PW), and preferably less than 3 times the pulse width, enhances power deliver per current provide (+IP or −In). Additional waveforms that can be used to deliver controlled power include square wave, sinusoidal, and noise. Frequencies between 1 Hz and 750 Hz may be preferred when combining thermal and electrical stimulation. Frequencies between 400 Hz and 14 kHz are preferred when mixing thermal and electrical stimulation. Frequencies from 12 kHZ to 100 kHz may be preferred to thermally dominant stimulation. The waveform frequency may also shift from one of these preferred ranges to another based on a schedule. For example, an embodiment of a fixed split schedule is 20 minutes in each frequency, for example 20 minutes in 100 Hz followed by 20 minutes in 20 kHz. An embodiment of a mismatched split schedule is 10 minutes or more at frequencies above 400 Hz or above 12 kHz, followed by 5 minutes or less at frequencies below 200 Hz or 600 Hz. Another mismatched split schedule is 30 minutes or more at frequencies above 100 Hz or above 10 kHz, followed by 10 minutes or less at frequencies below 100 Hz or 500 Hz. This is based on the slow kinetics of temperature changes as dictated by thermal stimulation algorithm 808 and molecular changes.

As mentioned, freedom to increase the amplitude and/or the duty cycle may be constrained by therapeutic, safety, or operational considerations. For example, some embodiments of the disclosed methods use current amplitudes that are sufficiently small that the patient does not perceive electrical stimulation. In other words, stimulation is below the perception threshold. Exceeding the perception threshold may not be desirable in some therapy modalities. In one embodiment the frequency is increased while maintaining power at a pre-targeted level until patient tolerability is acceptable. In this way temperature control is achieved while accommodating for subject tolerability. For example, a sinusoidal waveform may be used and frequency increasing while maintaining amplitude. Or a pulse waveform may be used with fixed amplitude, but duty cycle is increased as frequency is increased. In one embodiment, frequency is increased in steps of 500 Hz which balance significant steps in tolerability with incremental steps for hardware limitations. Frequency can begin a low range below 500 Hz, such as 50, 100, or 200 Hz, and then increase to above 1 kHz, such as 2 kHz, 10 kHz, 20 kHz, or 100 kHz. Frequency is then systematically tested in the intermediate frequency ranges. For example, a sequence may include 50 Hz, 2 kHz, 1 kHz, 100 Hz, 500 Hz. A sequence may include 20 Hz, 20 kHz, 10 kHz, 500 Hz, 800 Hz. A sequence may include 150 Hz, 100 kHz, 1 kHz, 100 Hz, 500 Hz. Each of these sequences may be supplemented with additional frequencies or modes as described here. Using temperature sensor and patient feedback they may be adjusted to optimize controller operation. Frequency exploration can be repeated every 1 week or every 6 months to test for changes in thermopoles to updated controller programming.

Temperature increase using thermopoles can implicate the strategy described here. The waveform applied across the selected electrode(s) by device hardware may achieve a prescribed power which may be controlled through RMS based on the thermal stimulation algorithm 808a/b. For example, three grades of control 1 mA, 2 mA, and 3 mA RMS may be provided. For current controlled devices RMS is the current RMS. The voltage thus adjusts accordingly based in impedance. To maintain voltage within require compliance the duty cycle may be greater than 30% and in some embodiments great than 60%. One such pulse pattern is 10 μs (first pulse), 10 μs (inter-pulse interval), 10 μs (reverse pulse) with a frequency of 30 kHz. Another such pule pattern is 1 μs, 1 μs, 1 μs with a frequency of 90 kHz. Another such pattern is an oscillation at 5, 10, 50, or 100 kHz which can be sinusoidal, square wave, trapezoidal, or noise based. Because tissue impedance decreases with frequency and because of device limitations, frequencies less than 100 kHz may provide electronic and tissue advantages. The voltage compliance that can be maintained may be 40 V and preferentially 20 V, for example, according to some embodiments. For any voltage compliance, the bioheat model 810 and thermopole algorithm 812 can be used to optimize the waveform applied accordingly. As the voltage decreases the duty cycle can be increased either by increasing pulse duration or by increasing frequency. In one embodiment, for each 10 V reduction in voltage, duty cycle is increased by 20% or 50% depending on tissue impedance. In another embodiment for each 10% reduction in voltage, duty cycle is increased by 8% or 16% depending on tissue impedance.

The relation between RMS intensity and tissue heating is a function of tissue properties and can be parameterized, for example, by impedance measurements and/or by measuring temperature increased due to prior RMS applications. The model parametrization, as executed by a microprocessor in a subject-specific basis during device use, can provide enhanced control or the control of RMS based on voltage limits. For frequencies greater than 5 kHz symmetric pulses may be used, based on (and subject to) electrochemical concerns. Frequencies bellow 500 kHZ may be preferred to minimize nonlinear tissue responses and interaction with other devices. When electrode size below 5 mm² is used, the relation of duty cycle with compliance can be adjusted such that for each 10% reduction in voltage, duty cycle is increased by 3% or 6% depending on tissue impedance.

It should be noted that the freedom to increase the duty cycle may be constrained because of charge buildup on the DC-blocking capacitors C1 and C2. As mentioned above and shown in the bottom trace of FIG. 10B, providing a constant current I between the electrodes causes the DC-blocking capacitors C1 and C2 to charge during the first pulse phases 1002a, which causes the voltages across them Vc1 and Vc2 to increase (I=C*dV/dt). The charges on the blocking capacitors contribute to the overall voltage drop through the system. Assume a compliance voltage VH is used to provide power to the DAC circuitry. The voltage drops through the circuitry to provide current through the tissue from E1 to E2 can be expressed as VH=Vp+Vc1+Vr+Vc2+Vn, which includes the voltage drops across the tissue (Vr), the DC-blocking capacitors (Vc1 and Vc2), and the selected PDACs and NDACs (Vp and Vn). As the DC-blocking capacitors charge, the total voltage drop can exceed the compliance voltage's ability to drive the prescribed current without increasing the compliance voltage VH, which decreases battery life.

Charge buildup on the DC-blocking capacitors occurs when the polarity of current is constant, for example, during the first phase of the biphasic pulse. Because the second pulse phase 1002b is of opposite polarity, Vc1 and Vc2 will decrease during the second pulse phases 1002b and return (ideally) to zero at the end of the second pulse phase 1002b. Thus, one way of providing more power through the tissue without overcharging the capacitors is to increase the frequency at which the polarities switch phases, that is, increasing the frequency of stimulation. Stated differently, higher frequency stimulation allows a greater effective duty cycle without overcharging the DC-blocking capacitors.

Referring again to FIG. 9, the microcontroller 814 can be configured to control the one or more temperature sensors 904. Under control by bus 114, a multiplexer 108 can couple or select signals of any of the temperature sensors at a given time. The analog signal from the temperature sensor(s) 904 can be converted to digital signals by one or more Analog-to-Digital converters (ADC(s)) 112. The ADC(s) may also reside within the control circuitry (i.e., the microcontroller 904), particularly if the control circuitry has A/D inputs.

Likewise, the microcontroller 814 can be configured to control the one or more thermal elements 902. Under control by bus 124, a multiplexer 128 can couple or select signals provided to any of the temperature sensors at a given time. The digital signals provided by the microcontroller 814 can be converted to analog signals by one or more DAC(s) 122. The DAC(s) may also reside within the control circuitry (i.e., the microcontroller 904), particularly if the control circuitry has A/D outputs.

It should be apparent that the implantable stimulation device 804 is configured to provide thermal stimulation to a patient's tissue via resistive heating within the tissue arising from electrical stimulation waveforms provided to the tissue via electrodes 16 and/or from thermal stimulation waveforms provided via thermal elements 902. The microcontroller 814 is configured to cause the thermodes (electrodes and/or thermal elements) to deliver stimulation waveforms calibrated to dissipate a controlled amount of power in the tissue, as informed by the thermal stimulation algorithm 808a/b (i.e., the bioheat model 810 in concert with the thermopole algorithm 812). The delivered power is controlled by controlling the amplitude, duty cycle, and frequency of the stimulation waveforms according to therapeutic considerations (e.g., sub-perception amplitudes) and within operational constraints of the implantable stimulation device 804 (e.g., without overcharging the DC-blocking capacitors 107).

The microcontroller 814 can be configured to automatically adjust the electrical and/or thermal stimulation waveforms based on readings of the one or more temperature sensors 904 to preserve temperature near the thermodes within a range and to automatically adjust stimulation parameters. For example, the thermal stimulation algorithm 808a/b may be configured to adjust the stimulation amplitude, frequency, and/or duty cycle based on signals received from the one or more temperature sensors, providing closed loop feedback for maintaining therapy. It should be noted, that the one or more temperature sensors can provide temperature readings even when no stimulation is being applied. The time-course for the stimulation parameters can be adjusted based on temperature readings and programmed objectives and can be determined based on the bioheat model and prior recordings, for example. For example, the applied waveforms can be adjusted after 10 or 30 minutes or based on monitoring and modeling under typical operational conditions. Under atypical operational conditions the waveforms may be adjusted on a less than 10 s or 1 s time frame. Examples of such atypical conditions can include an increase in measured temperature about a set threshold such as 38° C. or 40° C. or a rate of temperature change above a certain threshold such 1 degree per 10 seconds or 1 degree per 30 seconds. The control timing may be further modified using historical measured temperature changes during stimulation. This control timing can maintain temperature with sufficient stability to activate the described molecular therapy cascade.

According to some embodiments, baseline and periodic temperature variations can be determined and calibrated. For example, the patient's baseline temperature may vary based on time and/or other variables such as sleep, activity, pain intensity, circadian rhythms, etc. According to some embodiments, a user may sample baseline temperature changes in the absence of stimulation to determine how the tissue temperature fluctuates based on such variables. Once calibrated, the system can apply thermal stimulation that causes temperature changes superimposed on the baseline temperature changes. According to some embodiments, the system may seek to normalize the baseline temperature variation from a “pathological” temperature variation signature to a “normal” or “therapeutic” temperature variation signature (e.g., time course of temperature variation), which may be determined by the system. According to some embodiments, the baseline temperature readings may feed into the bioheat model, further refining the model.

The position of the lead(s) can be set by the bioheat model to optimize thermopole distribution relative to target. Specifically, the optimization processes described herein can be applied for multiple potential lead positions. The optimization can be applied to lead positions that vary by a point spread function of the thermopoles. For most applications, position increments of 1 mm or 3 mm will be accessed. Temperature measurements can be used during the lead implant to refine the bioheat model. In this way the bioheat model is updated at each position such that predictions about future positions are increased in accuracy. Conversely, the desired thermopole may be specified, and the optimal lead position predicted based on thermal optimization. The spatial increments evaluated can be 1 mm or 3 mm. One or more thermopoles may be used to constrain lead position. A thermopole may be targeted to the spinal cord white matter and a second thermopole targeted the spinal cord grey matter. Each thermopole can be assigned a target peak temperature, for example, a peak temperature of 0.5° C. at the white matter and 0.4° C. at the grey matter, or 0.8° C. at the white matter and 0.5° C. at the grey matter. A thermopole may be targeted to the epidural fat and a second thermopole targeted the spinal cord grey matter, for example, a peak temperature of 1° C. at the epidural fat and 0.4° C. at the grey matter or 1.5° C. at the epidural fat and 0.5° C. at the grey matter. These temperature differentials may optimize thermal based neuromodulation while being constrained by other practical factors.

At the end of the trial stimulation phase, a decision may be made whether to abandon stimulation therapy, or whether to provide the patient with a permanent stimulator device. Should it be determined that stimulation therapy is not working for the patient, the leads 14 or 15 can be explanted from the patient's spinal column 60 and incision 62 closed in a further surgical procedure. The decision to explant can be based on the performance based on the bioheat model and temperature sensors. If the temperature target is achieved, then therapeutic outcome may be forthcoming after a delay. In this case, an additional 2 or 6 weeks may be used. Specifically, subjects not showing a sufficient clinical response (i.e., pain reduction) may remain candidates for a permanent IPG 10 provided they presented a bioactive thermopole. An example of a bioactive thermopole can include a temperature rise of 0.1° C. or 0.5° C. at the spinal cord or 0.3° C. or 0.8° C. at the epidural fat. In subjects not exhibiting a sufficient clinal response and without a bioactive thermopole the bioheat model may inform new stimulation parameters extended the period before explant.

By contrast, if stimulation therapy is effective, a stimulation device can be permanently implanted in the patient as discussed above. (“Permanent” in this context generally refers to the useful life of the stimulation device, which may be from a few years to a few decades, at which time the stimulation device would need to be explanted and a new device implanted). The product lifetime may be adjusted based on the bioheat model and resulting thermopoles. The cycling of thermopole can extend the product lifetime. The short transition times are informed by bioheat models the thermal and electrical conductivity of tissue. The long transition times are informed by bioheat models including kinetics of the molecular changes underlying therapeutics outcomes. Transition from two stimulation modes, one with a peak temperature of great than 0.8° C. and on with a peak temperature of less than 0.3° C. at a target tissue can enhance product lifetime without cancelling therapeutic outcomes. The direct stimulation effects may be maintained across switching modes. Short transition time switching between ranges may occur every 1 to 40 minutes.

According to one embodiment, a short transition time of 15 minutes is used which can correspond to the needed time to achieve target temperature for given stimulating program (or mode). The mode, with distinct thermopoles, may be switched between every 15 minutes. For thermopoles targeting deep tissue, a corresponding switching time of 30 minutes can be used. Long transition time switching times between stages may occur every 6 hours to 15 days. Long transition times may be based on specific hours. According to one embodiment, mode 1 is activate from 6 AM to 10 PM and mode 2 is active from 10 PM to 6 AM. This or similar fixed schedules of long transition time correspond to activity periods. Activity periods may alter thermal demands. Short and long transition time may be interlaced. The switching time is adjusted, based on the bioheat model updated from temperature sensors. In one embodiment, a short-adjusted switching time of 5 to 10 minutes between to modes can be used. The time spent in each mode can be adjusted based on the desired temperature field. For example, mode 1 may be applied for 5 minutes and mode 2 applied for 10 minutes. Based on an updated bioheat model, mode 1 may be then applied for 10 minutes and mode 2 applied for 10 minutes. A short switching time of 1 to 40 minutes thus allows titration of thermopoles based on the temperature dynamics predicted by the bioheat models.

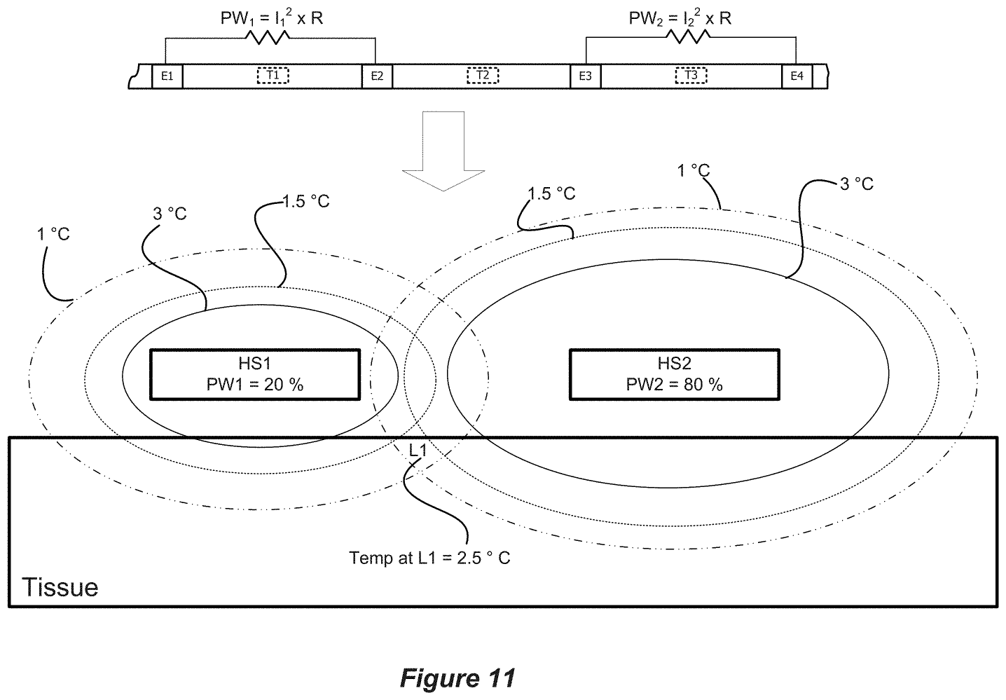

As mentioned above, aspects of the thermopole algorithm 812 determine appropriate stimulation parameters for providing controlled and directed thermal fields (i.e., thermopoles) within specific locations within a patient's tissue based on the bioheat models 810. Referring to FIG. 11, assume a clinician wishes to affect a temperature increase of 2.5° C. at a location L1 within a patient's tissue. The thermopole algorithm 812 can determine which thermode(s) to employ to generate the appropriate power at the appropriate locations to affect the prescribed temperature fields. The thermopole algorithm further determines the appropriate stimulation parameters.

To achieve the prescribed thermal stimulation, the thermopole algorithm uses thermal basis functions generated for individual thermodes and thermode combinations to create composite isotherms to spatially control thermal stimulation. The thermal basis functions model the thermal response of the tissue to various electrical stimulation parameters and can be based on modeling of the bioheat (e.g., finite element method (FEM) modeling), translation from RMS and/or active specific absorption rate (SAR) calculations, look-up tables, and the like. The thermal basis functions can also be based on, or refined based on, temperature readings from one or more of the temperature sensors.

In the example illustrated in FIG. 11, the algorithm determined that the prescribed temperature change at L1 can be affected by applying a first electrical stimulation using electrodes E1 and E2 and a second electrical stimulation using electrodes E3 and E4. Power PW₁ is dissipated into the tissue by the stimulation at E1/E2, based on the current Ii and the tissue resistance R. Likewise, power PW₂ is dissipated into the tissue by the stimulation E3/E4 based on the current I₂ and the tissue resistance.

The two pairs of electrodes E1/E2 and E3/E4 effectively provide two heat sources. The E1/E2 heat source is labeled HS1 and the E3/E4 heat source is labeled HS2 in the illustration. In the illustration, assume that of the total power provided to the tissue, HS1 provides 20% of the power and HS2 provides 80% of the power. In other words, PW2 is greater than PW1. FIG. 11 illustrates three isotherms associated with each of HS1 and HS2—one at 3° C. (solid lines), one 1.5° C. (dotted lines), and one at 1° C. (dashed/dotted lines). The isotherms associated with HS2 are further away from the center of the heat source, compared for those of HS1 because more power is dissipated at HS2. As calculated by the temperature algorithm, the isotherms overlap and enforce each other to provide a temperature increase of 2.5° C. at the location L1 within the tissue.

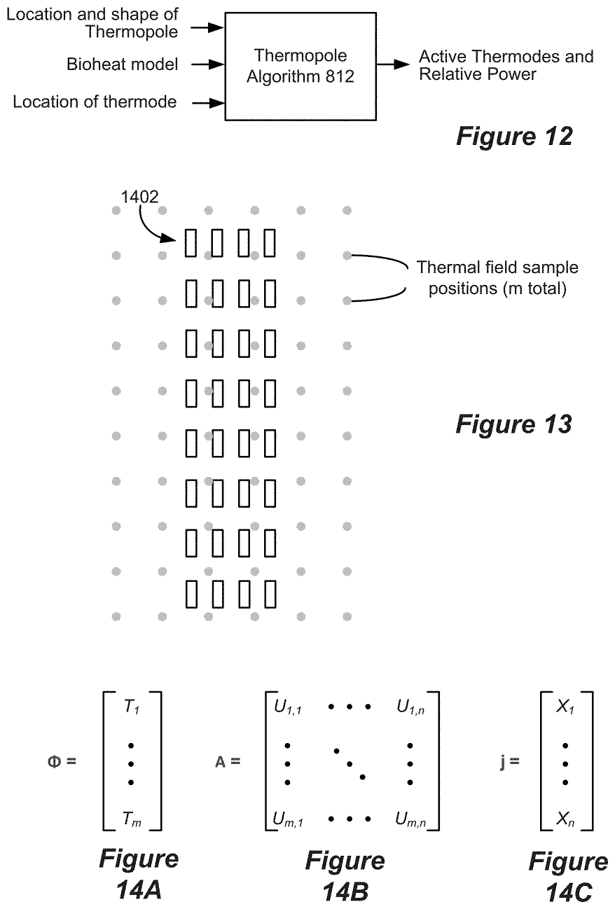

Thus, the thermopole algorithm 812 considers the thermode type, geometry, position, etc., and fractionates the power dissipated at the thermodes to achieve the prescribed thermopole, based on the bioheat model 810. The impedance between each active electrode pair can be determined or the impedance across may pairs of active and inactive electrodes can be determined to parametrize the bioheat model. For two thermodes, impedances at three electrodes may be measured. More generally, the minimum number of electrode pairs is the number of thermodes plus 1. When 4 thermodes are used, impedance is measured across five electrode pairs. The impedance can be measured while thermodes are active or in a separate calibration mode. Tissue undergoes a change in properties with increasing temperature. Measurement of impedance across different modes can be used to parametrize the bioheat model. The controller may step though programs increasing temperature at a target tissue by 0.05° C. or 0.1° C. increments until a target peak temperature is reached and the impedances may be measured across all relevant electrode pairs at each increment. FIG. 12 shows an example of the inputs and outputs of an embodiment of a thermopole algorithm 812, operable in the relevant external device 802, which may run automatically or upon a user selection in GUI 806. The inputs include the desired location of the thermopoles (which again need not correspond to physical thermode positions); the desired shape, i.e. magnitude and profile of the thermopole (which can be set in GUI 806); the bioheat model 810; and the location (and type and capacity) of the physical thermodes for example, available in the lead or array 1402 (FIG. 13).

According to some embodiments, the bioheat model can be used to evaluate the temperature field that would be generated as a result of stimulation at the thermopoles (i.e., if actual thermodes were present at those positions), and may take into account the different conductivities, thermal conductivities, and sizes of anatomical structures in the tissue, such as white matter, gray matter, cerebral spinal fluid, the dura, and vertebral bone in the area of the thermopoles, as described above and in the Examples. FIG. 13 shows an array of thermal field sample positions (m total) and an array of thermodes 1402 having associate thermal basis functions. Using modeling of the tissue, such as the bioheat model, a temperature Tm that would be induced at each of m thermal field sample positions in the tissue is determined that would result from thermal stimulation at the m sample position. The modeled temperatures at each of the m sample positions can be represented as a m×1 vector, φ (FIG. 14A).

The bioheat model is also used to determine temperatures Uₘₙ that would be induced at them sample positions as a result of stimulation using n physical thermode combinations. While the modeled thermode combinations can include any number of combinations of the thermodes, in one simple example, the n thermode combinations are binary combinations that are incremented along the thermode array. (Thus, in this example, n equals the number of thermodes in the electrode array minus one). The modeled temperatures Uₘₙ can be represented as a m×n transfer matrix, A (FIG. 14B). Thus, U_{1,1} comprises the temperature at sample position 1 simulating stimulation at thermode combination 1; U_{1,2} comprises the temperature at sample position 1 simulating stimulation at thermode combination 2; U_{2,1} comprises the temperature at sample position 2 simulating stimulation at thermode combination 1, etc. Any number of thermode combinations n and sample positions m can be modeled, which would increase the size of the transfer matrix A and promote higher solution accuracy, although a larger transfer matrix A is also more computationally difficult.

The thermode combinations that would induce thermopoles at the m sample positions that best match those generated as a result of stimulation at the thermopoles (φ) can be determined by solving for a vector j that minimizes the equation |φ−A*j |², where j comprises a l×n matrix indicating a weight Xn that each nth thermode combination plays in forming the desired thermal field. Such solution involves inverting the transfer matrix A (A⁻¹), such that j=A⁻¹*φ. The weights of the thermode combinations in vector j can then be summed to determine a physical thermode configuration—i.e., which physical thermodes should be active, as well as their relative power—to produce the thermopole's desired thermal field. In short, the output of the thermopole algorithm 812 determines a thermode configuration (active thermodes, their power) necessary to best produce the desired thermal field at the specified thermopole.

Regarding thermal stimulation using electrodes as the thermodes, i.e., via joule heating arising via electrical stimulation, it should be noted that temperature lead fields may be optimized in ways distinct and not obvious from designs addressing electrical stimulation (i.e., traditional and high frequency electrical stimulation/modulation therapy). This is because the temperature lead fields and electric stimulation lead fields may be distinct.

A comprehensive approach to stimulation efficacy and safety considers both temperature and electric fields as well as other electrode safety and device factors. For example, in creating stimulation pulse intensity or duration, decreases action potential thresholds and increases in temperature are an exponential factor of RMS. In one embodiment the inter-pulse duration is reduced to reduce electrical stimulation efficacy while maintained temperature lead fields. The inter-pulse interval can be less than 20 μs or less than 1 μs, for example. In another embodiment the pulse duration can be below 20 μs, or below 3 μs while pulse intensity can be increased by a proportional amount to maintain power, for example. One example of a preferred waveform is 20 kHz with pulse durations of 10 μs and an inter-pulse interval of 10 μs which achieves effective temperature fields whole controlled membrane polarization. Another example of a preferred waveform is a frequency greater than 40 kHz with a duty cycle greater than 80% or a frequency greater than 100 kHz with a duty cycle greater than 90%. Increasing frequency with a controlled duty cycle limits membrane polarization while controlling temperature lead fields. To control network activation while maintaining temperature lead fields, the pulse waveform may be altered while RMS or waveform power is maintained. In one embodiment the frequency jitters by 1-100 Hz while duty cycle is adjusted based on frequency to maintain power. An increase in frequency is associated with a decrease in duty cycle to maintain power. Significant jumps in frequency may be used. A frequency jump from 1 kHz to 10 kHz or 100 kHz can be implemented while maintaining RMS. The jump can be cycled every 1 second or every 1 minute.

Stimulation jitter or jumps can be combined with inversion of leading pulse polarity.

Whereas conventional stimulation depends on leading pulse polarity, temperature lead fields are independent of polarity. In one embodiment, the thermopole algorithm can determine an optimal electrode pair and leading pulse polarity that may correspond to perception, but then the leading polarity can be flipped. In this way the direct membrane polarization is a made less effective while stimulation temperature fields are maintained. Such a polarity flip may be accompanied by a frequency increase. Thus, electrical stimulation perception can be used to identify appropriate target tissues for thermal stimulation. Such techniques can be implemented in the “Search Mode” phase of implant fitting, as described in more detail below. For example, in the acute post implant programming stage, preferred perception may be identified for electrodes 1 and 2 on lead 1 with a polarity of the leading pulse cathodic from electrode 1 to 2 and a frequency of 50 Hz. Under such stimulation during the search phase, the 50 Hz waveform does not need to provide a high or controlled temperature field for this purpose of target identification. For the next stage the device is programmed for greater than 1 kHz or greater than 20 kHz with the leading pulse polarity anodic from electrode 1 and 2. In subsequent subject testing, the programmer may switch back to a 50 Hz frequency and if a new optimal electrode identified based on perception the 1 kHZ or 20 kHz waveform programmed accordingly. For differentiating the interrogation and secondary stages, a difference in frequency of greater than 20-fold or greater than 400-fold for the two phases may be preferred. Likewise, a difference in duty cycle of greater than 5-fold or greater than 60-fold may be preferred for differentiating the interrogation and secondary stages.

High frequency signals used to create thermopoles may be amplitude modulated to maintain thermopole peak and distribution while modifying secondary activation mechanisms. Such secondary mechanisms can include electric stimulation of nerves to fire action potentials or polarization of synaptic terminals to alter synaptic efficacy. It should be appreciated that thermopole modalities and electrical modalities may operate under different time regimes. For example, bioheat kinetics are typically greater than 60 seconds while membrane kinetic are typically less than 20 ms or less than 1 ms depending on the tissue target. As an example, a high frequency waveform (e.g., a sinusoidal stimulation at greater than 1 kHz) may be modulated at a lower frequency (e.g., less than 60 Hz). Examples include a 2 kHz sinusoid modulated at 10 Hz or a 10 kHz sinusoid modulation at 10 Hz. The average peak current or the average RMS current can be matched to the non-modulated waveform by enhancing the peak current. A square wave, trapezoidal wave, or other repeated waveform may be substitute for a sinusoid while accounting for the altered frequency content. The waveform, carrier frequency, and amplitude modulation frequency can be selected to create a thermopole and a region of influence based on a secondary activation mechanism. There regions may partially overlap. When using more than two pairs of electrodes, the carrier frequency applied to each may be different by the intended amplitude modulation to produce an interference zone. For example, a combination of 2 kHZ and 2.01 kHz produces a zone modulation at 10 Hz. This zone is distinct form the thermopole and therefore the controller can integrate both thermopole and secondary activation constraints. Thus, optimized intervention is obtained that different from that expected from temperature or secondary activation alone. For pulsed stimulation the frequency across electrode pairs can differ by greater than 5-10% or greater than 10-40%. This achieves a stimulation mismatch while minimal variation in pulse compression factor across thermodes. The phase between leading pulses across different electrodes may vary by the 1-3-fold of pulse width of the leading pulse. The titration of phase interferes with direct stimulation but not thermopoles. The phase may be constant or may include a jitter. The jitter may be 40-150% of the leading pulse width, for example. The jitter may thus be set to avoid significant change in temporal waveform while reducing consistency in direct stimulation by pulse convergence across electrodes. Direct stimulation depends on instant electric field distribution across target tissue. Thermopole remain unaffected as long as RMS is controlled. According to some embodiments, a noise-like pattern can be applied across electrode pairs. The noise may be constrained to minimize synergistic direct activation across pairs while controlling waveform RMS. White or pink noise may be preferred. The noise pattern may be constrained based on the stimulator electronics including the analog output stage. The bioheat model may select the frequency content of the signal based on the desired thermopoles and other programming constraints. The peak current applied at each pair may be mismatched. For example, the peak at one pair may be greater than 5-fold above the other pair to produce an asymmetric overlap of thermopole and secondary activation. Likewise, the peak current applied at one pair may be greater 1 mA above the other pair to produce an asymmetric overlap of thermopole and secondary activation. This difference may correspond to a temperature increase of about 0.5° C. More generally, the relative amplitude of the waveforms applied to each electrode pair can be adjusted by the controller to bias the secondary activation mechanism, while the average amplitude across both waveforms is adjusted for peak temperature control. In this case, the two pairs of electrode may be place on opposite side of the tissue target. This may be achieved by using two more leads implanted across the target. The use of more than two pairs allows to selection of multiple tissue targets each with a distinct thermopole vs secondary activation mechanism. The number of tissue targets is equal to the number of electrode pair minus one. For pulse stimulation with a rate greater than 1 kHz amplitude and modulation at 200 Hz or less is effective in maintaining thermopoles while engaging or modify secondary mechanisms. Pulses may be synaptic or asymmetric but maintain charge balance on a timescale less than 1 s. Frequencies of about 200 Hz or less used in conventional stimulation generally use waveforms that have weak thermopoles because the duty cycle cannot be increased sufficiently without sacrificing electrochemical stability. In contract, simulation at kHz or above can be achieved with acceptable duty cycles with amplitude modulation.

The lead position and waveforms provided can be optimized to produce a functional point spread function of the thermopole. The point spread function is the extended spatial temperature field that represents the bioheat response. In other words it is the spatial domain version of the bioheat response. The degree of spreading (blurring) of the thermopole is a measure of nervous system modulation. When two thermodes A and B are activated simultaneously, the resulting thermopole is the sum of the independently activated thermodes. The controller deconvolution of the point spread function and the thermopole-enhanced temperature field can be controlled, for example, to achieve a point spread function of greater than 0.5 mm. For example, according to some embodiments, the point spread may be 0.5 mm to 1.0 mm, or greater.

The point spread function varies with the bioheat transfer function and applied power. A lower power and closer electrode proximity result in a smaller point spread function, i.e., more focused thermal stimulation. For example, two electrodes separated by less than 5 mm, preferably less than 2 mm, provide moderate and high thermopole control when the RMS is below 5 mA. Two electrodes separated by less than 2 mm, preferably less than 1 mm, provide moderate and high thermopole control when the RMS is between 5 mA and 7 mA. Two electrodes separated by less than 1 mm, and preferably less than 0.5 mm provide moderate and high thermopole control when the RMS is between 7 mA and 10 mA. When feedback is used with one or more temperature sensors, the sensed temperature signals provide a statistical estimate of the bioheat transfer function for the controller. Inverse filtering the recoded signal can be used to constrain the controller and resulting point spread function. With the bioheat model the controller estimate can be improved using techniques such as Wiener deconvolution. The point spread function can be reduced 2-5-fold using temperature sensors. The point spread function can be reduced 1-3-fold using impedance measurements.

According to some embodiments, the thermal field should extend for a distance from the thermode(s) to reach targeted neural tissue. For example, thermodes may be placed in the epidural fat layers such that heat builds up in the fat and propagates to the neural target tissues, such as dorsal roots, spinal cord, etc., which may be 2 mm-6 mm away. According to some embodiments, at least a portion of the intervening material may comprise a material that has a lower thermal conductivity than the target neural tissue. For example, the intervening space may comprise a material with a thermal conductivity 3-fold to 50-fold less than the thermal conductivity of the neuronal target tissue. The low conductivity material may comprise tissue such as fat. According to some embodiments, the thermodes themselves may be encased in a material with low thermal conductivity, as mentioned above. The bioheat model and/or thermopole algorithm considers the thermode placement and thermal conductivity of the target tissue and intervening space in deriving the thermopoles invoked in the tissue. According to some embodiments, thermal stimulation can provide a temperature increase of 0.1 to 6.0° C., for example 0.5 to 4.0° C. in the targeted tissue. According to some embodiments, at least two thermodes may have an inter-thermode distance of 0.8 to 2.5 times the minimal distance between either of the thermodes and the target tissue. According to some embodiments, at least two thermodes have an inter-thermode distance of less than 1 mm and less than the minimal distance from either thermode to the target tissue. According to some embodiments, the thermopole(s) may be largely maintained over a period greater than 1 minute. For example, the thermopole(s) may be maintained for greater than 10 minutes or may be maintained for days or months.

According to some embodiments, the thermopole algorithm controls the microcontroller in accordance to the power law relationships contained in the bioheat model (see Equations 4 and 5 in the Examples) with a power between 1.4 and 3.5 or between 1.7 and 2.2. The power law relates the power applied to a thermode and the peak temperature in the thremopole. Because of the power law relationship, the thermopole algorithm may not use a linear model to adequately maintain temperature at the tissue target. According to some embodiments, a default power of 2 may be used. Individual difference in anatomy and lead placement, as well as tissue properties will affect the power law number. According to some embodiments, the power law is most effective for electrodes with a surface area great than 0.5 mm² and an inter-electrode distance greater than 1 mm. For example, information on lead impedance, or position, or anatomy may be used to determine the power. A limit on stimulation power may be set based on the power law. The power law may be supplemented by a multi-order polynomial or a look-up table. When more than two thermodes or more than two electrode pairs are used, each thermode or pair may be assigned a respective power law. When an optimal strategy is searched, a power law closest to 2 may be selected. For a power law greater than 2.5 the thermopole algorithm may limit changes in waveform to every 1 minute. For a power law greater than 1.8 the thermopole algorithm may limit changes in waveform to every 3 minutes. This is to account the difference in active and passive properties determining the power law and so potential tissue response transients.

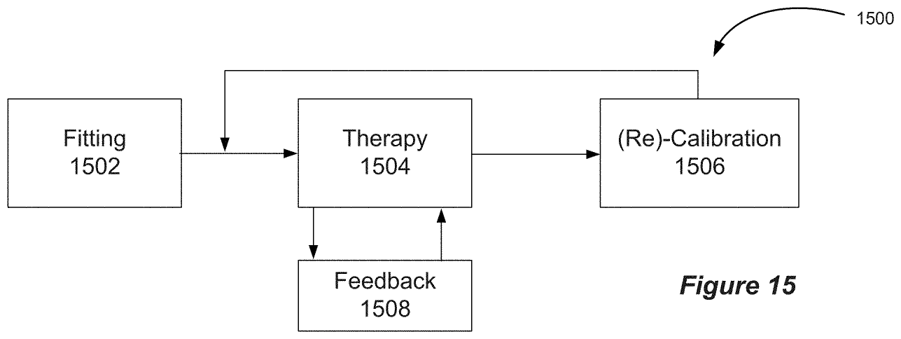

FIG. 15 illustrates an example of a workflow 1500 for delivering and controlling dosed and calibrated thermal stimulation. During a fitting procedure 1502, a user (typically a clinician) determines appropriate electrical and/or thermal stimulation that will best alleviate a patient's symptoms. Part of the fitting procedure 1502 includes determining which thermodes should be activated by the implantable stimulation device; the polarity of these active thermodes (if relevant); the amplitude of stimulation; (if stimulation is issued in pulses) the pulse width, frequency, the duty cycle (DC), and shape of the waveform (e.g., pulses); etc. for providing appropriate thermal stimulation. Initial fitting of a patient to determine a stimulation program that is effective usually occurs using a clinician programmer 90 (FIG. 3, FIG. 12), but fitting or stimulation program adjustment can also occur using any of the other external devices discussed above, such as a patient external controller 50 (FIG. 2). Fitting can occur both during an external trial phase as described earlier and after a permanent IPG 100 has been implanted.

Once the user has performed the fitting procedure 1502, thereby determining the appropriate thermal stimulation to apply to the patient, the external device transmits the appropriate parameters to the implantable stimulation device to provide the prescribed therapy 1504. The thermal stimulation algorithm 808b of the implantable stimulator device 804 may include programming configured to monitor, maintain, or adjust the stimulation parameters based on feedback 1508. For example, a particular temperature value or range may be prescribed for the thermal stimulation and the temperature algorithm 808b may adjust stimulation parameters to achieve that temperature value or range. From time to time, the user (either a clinician or the patient) may adjust or recalibrate 1506 the therapy using an appropriate external device.

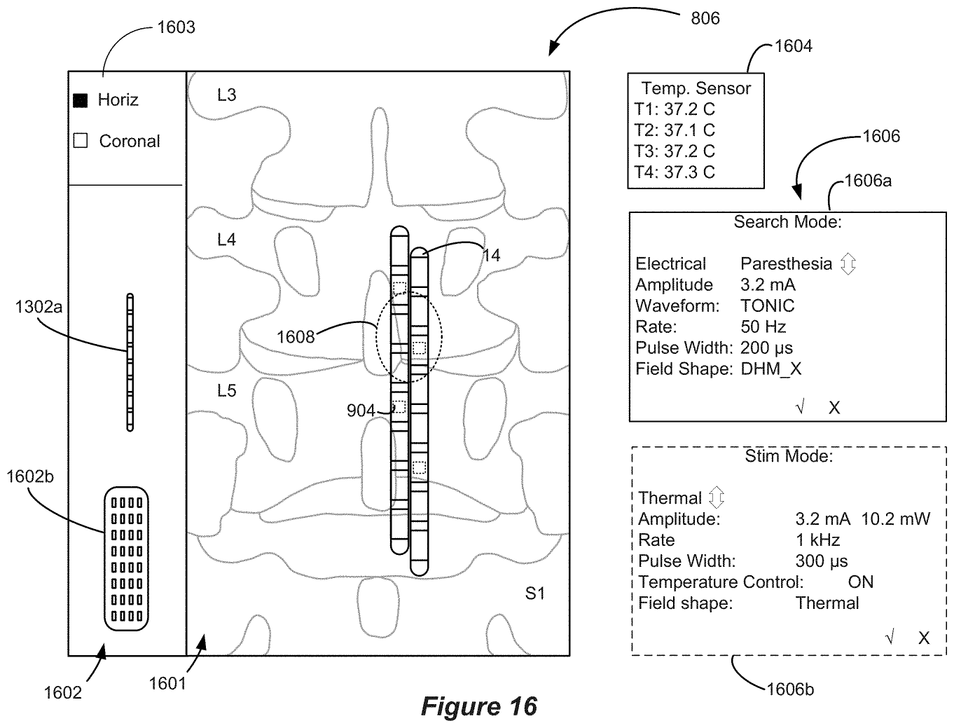

FIG. 16 shows a graphical user interface (GUI) 806 that can be used to set an electrical and/or thermal stimulation program for a patient as described above. GUI 806 allows a user to steer thermopoles around one or more electrode leads 14, which provides an automated and convenient means for setting and adjusting a thermal stimulation program. GUI 806 is rendered by execution of programming, including the execution of aspects of the thermal stimulation algorithm 808 within the external device 802.

GUI 806 may include a fluoroscopic image 1601, which shows one or more implanted leads relative to anatomical structures, such as vertebrae (L3, L4, L5, and S3 are shown). A user can select a graphical representation of the implanted electrode lead(s) from left side panel 1602, which includes representations of various types of leads such as a 1×8-electrode percutaneous lead representation 1302a, and a 4×8-electrode paddle lead representation 1602b. More than these two lead types and leads with different numbers of electrodes and/or other thermode types may also be represented. The fluoroscopic image 1601 may contain more than one lead representation, for example, left and right percutaneous leads, to match the number of leads implanted in the patient. Two percutaneous leads 14 are illustrated in FIG. 16. The user can select (e.g., by dragging) the appropriate lead representation(s) 1602 onto the fluoroscopic image 1601 and manipulate its size and orientation until it aligns with the implanted electrode lead in the fluoroscopic image 1601. Because the lead representations 1602 are programmed with appropriate thermode size, shape, and spacing for each of the leads, the positioning of a lead representation on the fluoroscopic image 1601 relates the locations of the electrodes to the anatomical structures in the image.

The GUI may include a view-selection window 1603, allowing a user to toggle between horizontal and coronal views. The horizontal view is selected in FIG. 16; the coronal view is illustrated in later figures.

The GUI 806 may include a readout 1304 for displaying temperature sensor readings of one or more temperature sensors 802 that are implanted in the lead(s) 14. The GUI 806 may also include one or more windows 1606 for setting and monitoring parameters related to stimulation, as described in more detail below. In FIG. 16, windows relating to a search mode 1606a (currently active in FIG. 16, as indicated by a solid outline) and a stimulation mode 1606b (currently inactive in FIG. 16, as indicated by a dashed line) are shown. The GUI 806 may display one or more contour lines 1608 indicating the region being thermally stimulated/modulated and/or being electrically stimulated/modulated. In FIG. 16, the contour line 1608 indicates a region of electrical stimulation, since the search mode window 1606a is selected and electrical stimulation (in paresthesia mode) is being applied.

As described above, an aspect of the fitting process 1502 is to determine proper location at which to apply electrical and/or thermal stimulation. To assist the user in locating an appropriate stimulus location, the GUI 806 can include a search mode 1606a, which employs waveforms and electrode configurations to find spinal levels related to patient pain. For safety reasons, electrical waveforms may be used in the search mode to avoid burning the patient. Various waveforms may be used. For example, the search mode may be employ a paresthesia waveform (as in FIG. 16), wherein the tonic waveforms are applied and the electrodes (or electrode combinations) are scanned to identify stimulation locations where paresthesia masks pain. As described above, once an appropriate placement is located based on paresthesia, the waveform may be altered, for example, by inverting the polarity of the leading pulse and/or altering the frequency, to provide thermal stimulation. Alternatively, sub-perception waveforms may be applied. One or more temperature sensor readings 1604 may be employed during the search mode to find (or avoid) applying stimulation at regions of inflammation (identified based on increased temperature at that location).

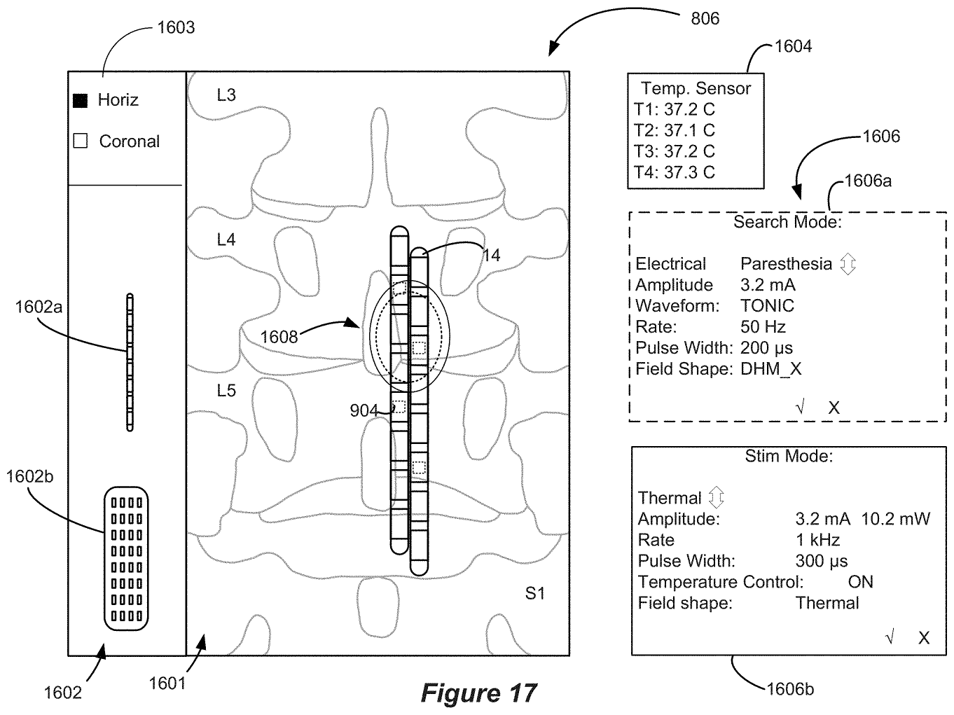

Once electrode positioning is determined in the search mode, the GUI 806 can be toggled to stimulation mode (currently active in FIG. 17, as indicated by solid line). In FIG. 17, thermal stimulation is being adjusted, as indicated in the “Stim Mode” box 1606b. In stimulation mode, the user can specify parameters for electric and/or thermal stimulation. In the case of electric-based thermal stimulation, current amplitudes can be translated into RMS power transmission, as described above. Alternatively (or additionally), power from other thermode types can be set. The GUI can include contour lines 1608 indicating temperature isotherms, i.e., indicating thermopoles, based on the bioheat model. According to some embodiments, a user may draw, or otherwise indicate a region to be heated to a specific temperature and stimulation will proceed until the temperature sensors detect that the indicated region has reached the target temperature. As explained above, the user can select thermopoles and the thermopole algorithm and/or bioheat model can perform the heating fractionalizations amongst the thermodes. Estimates of the heating due to stimulation may appear on the horizontal view or on the coronal view of the GUI 806. According to some embodiments, the GUI may be configured to represent a series of time-based temperature maps.

As mentioned above, heat readings and thermal dosing may be based on temperature sensor readings. Additionally (or alternatively), the determination of optimal heating may be based on other biosensors or biofeedback, for example, LFP sensors configured to detect neurological or other activity indicating adequate or excessive heating. Optimal heating may also be determined based on patient heart rate.

The GUI may also include warnings that can be issued if heating becomes too extreme. For example, the GUI may present a warning if heat exceeds a safety level and/or if heat exceeds a level corresponding to optimal HSP dosing. If excessive heating is detected, then stimulation amplitude can be decreased. Also, warnings may be issued if a user wishes to stimulate regions that are already at an elevated temperature, for example, due to inflammation. Likewise, as low-level heating is marker of inflammation, thermal probe/temperature sensors on the lead may also be used to map target tissue temperatures before and/or after stimulation and display a temperature map of the target tissues for diagnostic/prognostic purposes.

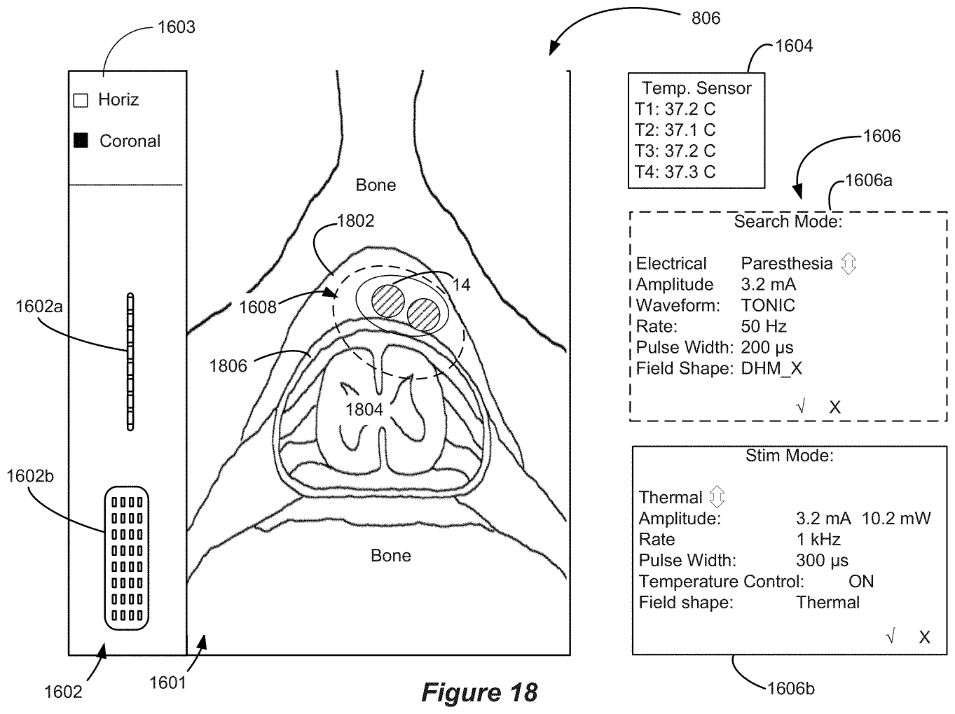

FIG. 18 shows GUI 806 wherein coronal view is selected. The electrode leads 14 are shown in an end-on perspective within the epidural space 1802. As in the horizontal view, the coronal view can include contour lines 1608 indicating temperature isotherms. The coronal view may include representations of spinal cord tissue 1804, dura 1806. It should be noted that spinal cord nervous tissue may not be the only heating target. Heating epidural fat and dura may produce anti-inflammatory effects via local HSP expression.

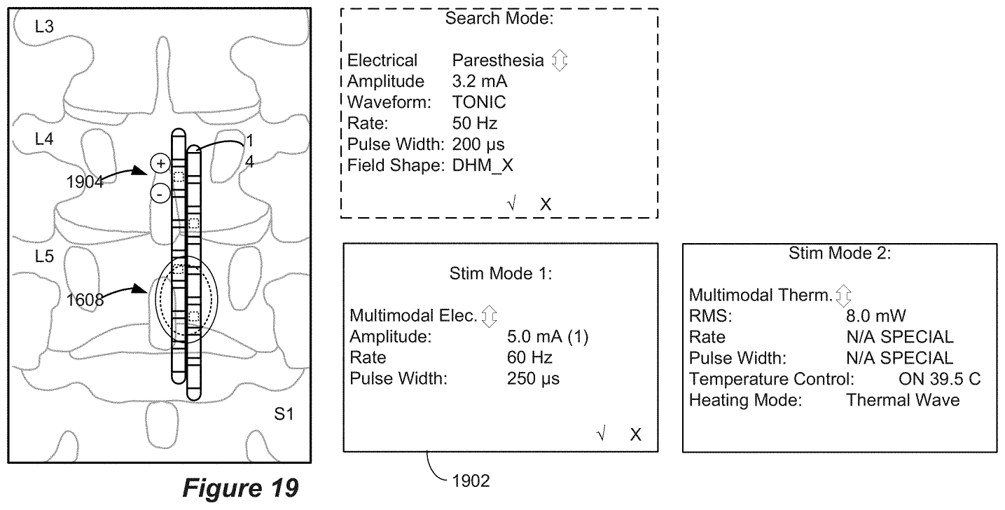

FIG. 19 illustrates aspects of an embodiment of the GUI configured for configuring multimodal stimulation, in this case thermal and electrical stimulation at area 1904. In addition to the aspects already discussed, the GUI can include additional windows, such as window 1902, for configuring additional modes of stimulation. Time courses for both the thermal and electrical stimulation may be configured. For example, at the induction of therapy and/or with lower total heating at area 1608, gate-controlled paresthesia-based stimulation may be applied at area 1904 using high amplitude, rate, pulse width, etc., to engage neural mechanisms while heating takes effect. At higher temperatures and/or if the patient reports feeling relief (for example, measured using a remote, app, or other patient feedback mechanism), the gate controlled stimulation at area 1904 may be dialed down to provide supplementary therapy but to also minimize paresthesia, power consumption, and/or unwanted effects.

Once appropriate stimulation parameters have been identified using the GUI 806 and the external device, the implantable stimulation device can be configured to run the parameters during ongoing therapy, with temperature and other feedback, as described above. According to some embodiments, the electrical parameters used during therapy achieve the therapeutic benefit due to thermal modulation of the neural elements while remaining below the patient's perception threshold. Thus, the embodiments provide sub perception therapy.

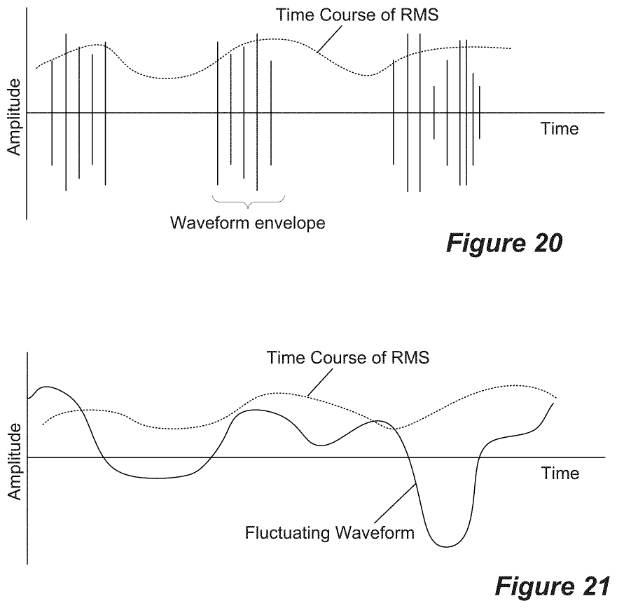

Embodiments of the GUI 806 can include displays and controls for setting time-course aspects of thermal stimulation and for deriving appropriate thermal stimulation waveforms to achieve and/or maintain the evolution of thermopoles over time. For example, an optimal stimulation burst pattern as a function of time may be derived to maintain a constant heating profile or for a pattern of heating. FIG. 20 illustrates a time course of RMS power for a pattern of waveform envelopes. According to some embodiments, the user may select a desired RMS time course. According to other embodiments, the user may select a temporal temperature profile. Still alternatively, the user may select a time course that is informed based on biofeedback (e.g., heart rate, accelerometer, metabolism, blood flow tracker, etc.) and the appropriate waveforms are determined. Pattern features (e.g., burst characteristics) can be adjusted to match an RMS template.

According to an alternative embodiment, a user may select a continuous, constantly fluctuating waveform, customized to hold a time-varying RMS for temperature control, as illustrated in FIG. 21. The waveform may be charge-balance over the appropriate interval.

According to one embodiment, the GUI offers operational models. One or more interrogation modes may activate the device in a manner intended to provide information for the bioheat model. Interrogation modes offer different time courses. For example, a time course of 1-30 seconds may provide an impedance-based interrogation mode. A time course of 5-40 minutes may allow for a temperature increment-based interrogation mode. The results of the interrogation mode(s) may update the bioheat model and so the intervention programming interface including the thermopole. In the intervention programming mode limits based on safety and other considerations are provided. These may be hard limited or allow the programmer a limited range of flexibility. These can include maximum frequency, maximum amplitude, maximum charge per phase, maximum tissue temperature, maximum target tissue temperature. The projections of both electric field and temperature field may be provided. These may vary independently based on lead programming. For example, electric field can be represented as neuromodulation efficacy. For the case of increasing frequency while maintaining duty cycle, the electric field neuromodulation efficacy may decrease while the temperatures field will be unchanged. In one application, the electric field in a region of interest may be set to ineffective levels while the temperature field may be set to an effective level producing a thermopole based neuromodulation. In one embodiment, the electric field in a region of interest may be set to effective levels while the temperature field is set to an effective level producing a thermopole and direct stimulation-based neuromodulation. The inter-electrode may be titrated changing the relative efficacy and depth of penetration of temperature fields and electric fields. With an optimal lead waveform set options can be provided for stimulation based on modes. One mode may be used with a fixed pattern until changed by the programmer. More than one mode may allow for change from one pattern to another based on a fixed period. For example, the programmer may select a first waveform with effective temperatures but ineffective electric field and then an additional mode with effective temperatures and effective electric fields. The transition between modes can be based on a fixed schedule such as 30-160 minutes per mode. The transition between modes can be controlled by an operator switch which may be available to the patient. The patient, using a remote app or remote control or other external device may switch between the two modes. For example, the patient may select one mode where paresthesia is absent corresponding to the first a waveform with effective temperatures but ineffective electric field and may switch to the second mode to transiently increase efficacy. For each mode, a range of intensities may be provided, such that after selecting a mode, or automatically switching to a mode, a user can adjust the peak amplitude of that mode.

Temperature sensor(s) may record and display readings on the GUI. These measurements may be overlaid with the bioheat model projections. The measurements may shape the bioheat model predictions where the measurements constrain the predicted temperature for a given RMS to the location of the sensor in the GUI map. A time series of temperature measures over time in the GUI may also be provided which can be correlated with changes in device programming. For example, the programmer may adjust the stimulation parameters to bring the temperature in a target region to a desired value. If a stimulation waveform is fixed for a sufficient time, such as 10minutes or 20 minutes, the temperature time series or map can reflect a steady state. For shorter times, this changes in temperature may reflect a bioheat dynamic. The bioheat model may predict the target temperature reached at steady state. With this GUI the programmer may adjust waveforms before steady-state is necessarily achieved. The dynamic bioheat model thus may support dynamic programming. Should the bioheat model predict a temperature or other transient beyond predefined safety limits a warning may be provided. In addition, the waveform may be automatically adjusted to prevent the temperature from attaining the safety limit. For example, the operator may set a program that after 1-minute increases temperatures by 0.5° C. and the bioheat model may predict a steady state temperature rise by 5° C. thereby triggering an alarm and an automatic reduction in stimulation RMS. The bioheat model may allow prediction of temperature increases across all tissue based on limited location of sensors. The number and position of sensors may be designed to maximize predictive value. Based on a specific rate of temperature increase the bioheat model and controller may also stop all stimulation. A warning may then be provided. Because the bioheat model is initiation parametrized based on prior experimental recording, the above regime may also operate without a temperature sensor based on bioheat predictions. The interrogation mode increases the accuracy of the bioheat model including the thermopole matrix. The temperature may be presented in false color or as lines.

The following example is included to illustrate embodiments of the methods disclosed herein. It should be appreciated by those of skill in the art that the techniques disclosed in the examples which follow represent techniques discovered by the inventors to function well in the practice of the disclosed methods. However, those of skill in the art should, in light of the present disclosure, appreciate that many changes can be made in the specific embodiments which are disclosed and still obtain like or similar results.

### EXAMPLES

The disclosed examples illustrate modeling for predicting the degree of tissue temperature rises driven by SCS joule heat, and characterizes the role of SCS waveform (including frequency, pulse width, and amplitude) and tissue properties. Temperature increases around an experimental SCS lead in a bath to verify a finite-element-model of SCS joule heat were determined. The dependence of temperature rise only on the power of the stimulation waveform, independent of other parameters was confirmed. Temperature increases during conventional and kHz-SCS at the dorsal spinal cord under passive and active bio-heat conditions in a geometric human spinal cord FEM model were predicted.

Method

Saline Bath Phantom

Thermal and electrical conductivity measurements taken to verify the general heat transfer model were performed in a cylindrical glass container (diameter: 90 mm and height: 130 mm) with three varied NaCl concentrations (154 mmol/L, 34.22 mmol/L, and 3.42 mmol/L (approximating cerebrospinal fluid, meninges, and epidural space respectively). A thermal conductivity meter (Therm Test Inc., Canada) and an electrical conductivity meter (Jenco Instruments, Inc., San Diego, Calif.) measured the thermal and electrical properties of the saline solutions at 37° C. (core spinal cord temperature approximation). The measured corresponding conductivity values for each molar concentrations were: electrical conductivity (σ): 1.62 S/m, 0.47 S/m, and 0.047 S/m; and thermal conductivity (κ): 0.6268 W/(m·K), 0.6317 W/(m·K), and 0.6319 W/(m·K) respectively.

In Vitro Stimulation

For the saline bath experiments, an experimental polyurethane SCS lead with 4 Platinum/Iridium electrode contacts (1.35 mm electrode diameter, 3 mm electrode length, 1 mm inter-electrode spacing) was placed at the center of the cylindrical container. The cylindrical container was then immersed in a temperature-controlled water bath (280×160×150 mm³) maintained at ˜37° C. and baseline temperature was stabilized for >60 minutes. Three different waveforms, namely sinusoidal, square, and a symmetric charge-balanced biphasic pulse waveforms mimicking the characteristics and parameters of clinical SCS waveforms (described by leading pulse duration, inter-pulse interval, recovery pulse duration), were generated using a function generator (AFG320, Tektronix, Beaverton, Oreg., USA). The generated waveforms were passed through a custom designed high-bandwidth linear current isolator to the experimental SCS lead. (Distal) Electrode contact 1(E1) and (proximal contact) 4 (E4) of the experimental SCS lead were energized for all saline bath experiments. Tested stimulation intensities were 1-7 mA (peak) using rates of 0.1 KHz to 10 KHz. Only for phantom verification, biphasic rectangular waveform pulse widths of each phase (40 μs) and interphases (10 μs) were kept constant such that the duty cycle increased directly with stimulation frequency.

Temperature Measurement and Analysis

A fiber optic temperature probe (STS Probe Kit, LumaSense Technologies, Inc. CA, USA) sensed by a fiber optic thermometer (±0.1° C. accuracy at calibration temperature, m600 FOT LAB KIT, LumaSense Technology, CA, USA) was positioned in the proximity of E4 to measure temperature increases during stimulation. The peak temperature change was measured in the bath radially from E4 (1 mm, 2 mm, 3 mm, and 4 mm) during stimulation as a function of peak stimulation amplitudes (1-7 mA), over a range of stimulation frequencies (0.1 KHz, 1 KHz, 5 KHz, 10 KHz, and 20 KHz) for sinusoidal, square, and SCS pulsed waveforms. Measured temperature was digitized using TrueTemp data acquisition and graphing software (60 samples/measurement and 1 second measurement interval, LumaSense Technologies, Inc. CA, USA). Temperature was normalized with respect to the initial temperature (˜37° C.), which was considered baseline.

Computational Models and Solution Method

Bioheat Model of Spinal Cord

Human spinal cord was simulated as a computer-aided design (CAD) derived model comprising seven compartments namely vertebrae (lower thoracic region, T8-T11), intervertebral disc, surrounding soft-tissues (minimally perfused), epidural fat, meninges, cerebrospinal fluid, and spinal cord (white matter and grey matter combined; FIG. 2). An MRI model may be developed using similar techniques. The dimensions of the individual tissues, modelled as isotropic homogenous volume conductors, were based on human cadaveric spinal cord from prior studies. The diameter of spinal cord with dorsal roots was fixed (spinal cord, 6.4 mm; dorsal roots, 0.5 mm) and the thickness of the adjacent tissues were: CSF, 2.0 mm; meninges, 0.5 mm; and epidural fat 1.0 mm. In situ, the diameter of the spinal cord varies along the vertebral column. Two SCS clinical leads were modelled and placed epidurally in a minimally staggered bilateral fashion (SCS Lead 1, 1 mm distal to the mediolateral midline at T8; SCS Lead 2, 0.5 mm away from SCS Lead 1 and proximal to the mediolateral midline at T9; FIG. 2A2). Only the first SCS lead was energized; the second lead was passive, positioned to mimic a clinical placement, and used to assess the impact of the presence of a passive lead on heat dispersion. The finite element method (FEM) model was solved using Pennes' bioheat equation governing joule heating during electrical stimulation (Laplace equation for electrostatics (∇(∇σV)=0 where V is potential and σ is conductivity), metabolic heat generation rate (Qₘₑₜ), and blood perfusion rate (ω_{b}) in the tissues as mentioned below:

ρCₚ∇T=∇·(κ∇T)−ρ_{b}C_{b}ω_{b}(T−T_{b})+Qₘₑₜ+σ|∇²| (1)

where ρ, Cₚ, T, σ, and κ represent tissue density, specific heat, temperature, electrical conductivity, and thermal conductivity respectively. Biological properties of blood such as density (ρ_{b}), specific heat (C_{b}), and temperature (T_{b}) were assumed constant in all vascular spinal tissues (vertebrae, meninges, spinal cord) and the corresponding values were 1057 kg/m³, 3600 J/(kg·K), and 36.7° C. respectively. Blood perfusion rate (ω_{b}) values were tissue specific and were in the range of 0.0003-0.008 s⁻¹. In spinal tissues, metabolic activities due to local spinal cord metabolism and enhanced metabolism in response to SCS generates thermal energy. Blood circulation also plays a significant role in transporting thermal energy across the spinal tissues through convection. The blood temperature in the spinal tissues was considered to be 0.3° C. less than core spinal cord temperature (37° C.). How the interaction between metabolic heat generation and blood perfusion modulates kHz-SCS induced temperature increases was investigated. Prior to the application of kHz-SCS, the metabolic heat generation rate required to balance the initial spinal cord temperature was calculated using equation (2) for the aforementioned perfusion rates as:

Qₘₑₜ=ρ_{b}C_{b}ω_{b}(T−Tb) (2)

where T and Tb are initial spinal cord and blood temperature. The calculated Metabolic Heat Generation (MHG) and the corresponding Blood Perfusion (BPer) values were given as; spinal cord and meninges (Qₘₑₜ, 9132 Wm⁻³; ω_{b}, 0.008 s⁻¹), vertebrae (Qₘₑₜ, 342 Wm⁻³; ω_{b}, 0.0003 s⁻¹), and minimally perfused soft-tissues (Qₘₑₜ, 457 Wm⁻³; ω_{b}, 0.0004 s⁻¹). The balanced Qₘₑₜ values were approximated based on prior experimental measurements.

Unless otherwise indicated, mimicking clinical montages and waveforms, electrode contacts E1 and E3 of the clinical SCS Lead 1 in a bipolar configuration (8 mm center-to-center electrode distance) were energized. Maximum temperature increases by conventional and kHz-SCS using rectangular waveforms for varied peak amplitudes (1, 2, 3, 3.5, 4, 5 mA), frequencies and pulse widths (50 Hz (200 μs), 100 Hz (200 μs), 1 KHz (40 μs and 100 μs), 5 KHz (40 μs), and 10 KHz (40 μs) were predicted and compared between active (bioheat) and passive heating cases at three different locations namely, at the distal edge E3 of the clinical SCS Lead 1 (˜0.01 mm from the surface of the lead), at the proximal surface of the dorsal root to the SCS lead, and at the surface of spinal cord (˜3.5 mm radial from the E3 electrode).

Boundary and Initial Condition

To model each stimulation waveform, corresponding static RMS values were applied (see phantom and model Results for justification). The accuracy of RMS intensities calculated analytically for a given intensity, frequency, and pulse width (see equation 3) were confirmed experimentally by stimulation across a resistive load (1 KΩ) with voltage acquisition using a digital mixed signal oscilloscope (MSO2024, Tektronix, OR, USA, ±(100 mv+3% of threshold)), a DAQ (NI PCI 5922, National Instruments, TX, USA, ±500 ppm (0.05%) of input+50 μV), and a digital multimeter (DMM 7510 7½ Digit Graphical Sampling Multimeter, Tektronix, OR, USA, ±60 ppm 0.0014% of input). The error in calculated versus measured RMS values was less than 5%.

$$\begin{matrix}{I}_{RMS} = \sqrt{\frac{1}{T} {\int }_{0}^{t} {I ( t )}_{peak}^{2} dt} \\ = {I ( t )}_{peak} \sqrt{\frac{t}{T}} \\ = {I}_{Peak} \sqrt{D}\end{matrix} \qquad ( 3 )$$

where I_{Peak} is the peak bipolar stimulation intensity, I_{RMS} is the corresponding RMS value, T is the pulse duration, t is the pulse width, and D is the duty cycle.

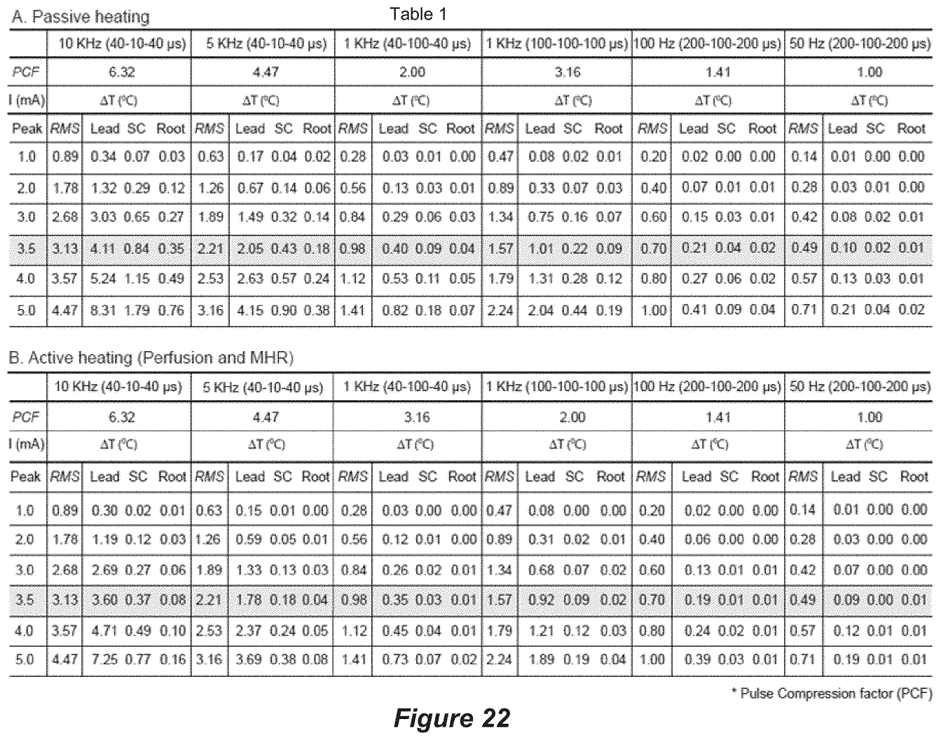

A static inward normal current density (Jorin, RIO corresponding to the stimulation current intensity (I_{RMS}, Table 1, FIG. 22) was injected through E1, and E3 was set as the return (producing a bipolar configuration). The electrical and thermal conductivities of the electrode contacts and the inter-electrode spacing were 4×10⁶ S/m and 31 W/(m·K), and σ=1×10⁻¹⁵ S/m; κ=0.0262 W/(m·K) respectively. The outer boundaries of the spinal cord and the surrounding tissues were considered electrically insulated.

For the thermal boundary conditions, the temperature at the outer boundaries of the spinal column was fixed at core body temperature (37 ° C.) with an assumption of no convective heat loss to the ambient temperature, no convective gradients across spinal surrounding tissues, and no SCS-induced heating at the model boundaries. The initial temperature of the tissues was assumed to be 37° C., and thermo-electrical properties of biological tissues were based on average literature values. Intravertebral disc (σ=0.830 S/m; κ=0.49 W/(m·K), epidural fat (σ=0.025 S/m; κ=0.21 W/(m·K), and csf (σ=1.65 S/m; κ=0.57 W/(m·K) are avascular, and therefore have no BPer and MHG, whereas the other remaining tissues are vascularized and have BPer and MHG as listed: soft tissues (σ=0.15 S/m; κ=0.47 W/(m·K), ω_{b}=0.0004 s⁻¹, Qₘₑₜ=457 Wm⁻³), vertebrae (σ=0.01 S/m; κ=0.32 W/(m·K), ω_{b}=0.0003 s⁻¹, Qₘₑₜ=342 Wm⁻³), meninges (σ=0.368 S/m; κ=0.44 W/(m·K), ω_{b}=0.008 s⁻¹, Qₘₑₜ=9132 Wm⁻³), and spinal cord (σ=0.126 S/m; κ=0.51 W/(m·K), ω_{b}=0.008 s⁻¹, Qₘₑₜ=9132 Wm⁻³). When indicated, these “standard” tissue values were manipulated by either 1) doubling or halving the electrical and/or thermal conductivities of a given compartment, or 2) by substituting properties across compartments.

Saline Bath Phantom FEM

SCS saline bath phantom was modelled using equation (1) while eliminating the biological tissue parameters. The FEM Phantom model was parameterized based on the dimensions, conductivity, and initial temperature of the experimental set-up. As tested, one SCS experimental lead centrally placed in a saline bath phantom was simulated. For the electrical boundary conditions, a normal RMS current density was applied at E4 (anode) and return at E1 (cathode). The outer boundaries of the bath were considered electrically insulated. For thermal boundary conditions, the external boundary temperature and the initial temperature of the bath were fixed at 37° C.

Model Construction and Computational Method

Human spinal cord and saline bath phantom models were CAD derived and imported. The entire volume of the spinal tissue and the electrode assembly was 83.0×74×108 mm³. Prior to the segmentation, tissues were resampled to have an isotropic resolution of 0.2 mm³. Resampled images were segmented into seven tissues compartments along with the T8-T11 positioned SCS lead assembly using a combination of automatic and manual segmentation filters. Using a voxel-based meshing algorithms, an adaptive tetrahedral mesh was generated. The final model size resulting from multiple mesh densities refinement contained approximately 4,600,000 tetrahedral elements for the full anatomy of spinal cord model and approximately 320,000 tetrahedral elements for the saline bath model. The meshes were imported to computationally solve the FEM model. The SCS model was solved for both passive heating (joule heating, without BPer and MHG) and active heating (bioheat, with BPer and MHG) conditions. The baseline temperature gradient for the active heating case was predicted by first solving the heat transfer model in the absence of electrical stimulation. In passive heating, the baseline temperature gradient was set to zero. The Saline bath model was solved only for passive heating condition. Both phantom and SCS models were solved under steady state assumption and corresponding temperature increases and field intensities were quantified. Heat flux and field intensity streamlines (seeded at selected tissue boundaries and proportional in diameter to the logarithm of corresponding magnitudes) were plotted to illustrate the overall distribution across tissues.

Statistics and Analysis

Normality test on temperature increases were conducted using Lilliefors corrected K-S test statistical test. A two-way repeated measure analysis of variance (ANOVA) was used to access the statistical differences in AT across different tested conditions (stimulation intensity, waveforms, frequencies, conductivities). A critical value (p)<0.01 was accepted as a statistical difference between the groups. Further significance between groups were verified using Post hoc Scheffe's test (corrected multiple comparisons). The statistical relations between the experimental data the FEM data was evaluated through a linear regression.

A power law shows super-linearity between the RMS and temperature increases, using a linear least squares fitting technique derived by Gauss and Legendre with a power function given as:

ΔT=A*RMS^{β} (4)

where ‘β’ is the power, and ‘A’ is the proportionality constant. The value of ‘β’ determines the category of the relationship (β=1, linear; β>1, super-linear; β<1, sublinear). Formulating the power function further on a log-log scale yields:

ln(ΔT)=ln(A)β*ln(RMS) (5)

Equation (5) is a straight line with a slope ‘β’ and a y-intercept of ln (A). Linear least square fit of the logarithmic data yields the correlation (r²)

Pulse Compression Factor per stimulation intensity (PCF) captures the increase in RMS of a High-Rate waveform (RMS_{High-Rate}) compared to a conventional 1 mA peak 50 Hz 200 μs pulse-width waveform (RMS₅₀):

RMS_{High-Rate}=Iₚₑₐₖ*PCF*RMS₅₀ (6)

PCF=10*\sqrt{Pw*f} (7)

where ‘Pw’ and ‘f’ are pulse width (sec) and frequency (Hz) for a given High-Rate waveform.

Results

Phantom Measurements and Model Verification

A specially designed chamber as described above was used to quantify temperature increases around an experimental SCS lead in a saline bath using varied waveforms. A micro-manipulator mounted optical temperature probe mapped steady-state temperature increases during stimulation with varied waveforms. As predicted by the FEM, temperature increases when applying a 10 KHz symmetric biphasic pulsed waveform at 5 mA peak intensity in a low conductivity saline phantom was maximal near energized electrodes and decreased with radial distance. In separate experiments, salt bath conductivity was varied by saline concentration. The main effect of saline bath conductivity and stimulation intensities (1-7 mA peak sinusoidal) was significant (F(2, 105)=218.95 p<0.01 and F(6, 105)=42.03, p<0.01). The interaction between these factors on ΔT was also significant; (F(12, 105)=19.88, p<0.01). Temperature increases were measured to be significantly greater in the lower saline bath conductivity (0.047 S/m) than in the other two saline bath conductivities (0.47 S/m and 1.62 S/m; Post-hoc pairwise comparison). Across different saline conductivities at different sinusoidal frequencies, the measured temperature increases were significant; F (2,75)=256.25, p<0.01. ΔT was higher at lower conductivity saline bath.

Temperature increased by up to ˜1° C. with stimulation amplitude during stimulation using all 10 KHz waveforms (symmetric biphasic pulse, square, sinusoidal). In addition, when considering only peak intensities, higher ΔT was observed during stimulation using pulsed and square waveforms versus the sinusoidal waveform (F (2,105)=41.14, p<0.01). However, this effect was found to be directly related to the RMS of the waveform and not to the specific shape of the stimulation waveform (F (2, 75)=1.11, p>0.01). The polarity of the leading pulse does not influence temperature in contrast to direct stimulation because of this feature.

In a separate series, temperature increases were measured across varied frequencies for all waveforms (symmetric biphasic pulse, square, sinusoidal) in a low conductivity saline bath with 5 mA peak current intensity (corresponding RMS: sinusoidal waveform, 4.95 mA; square waveform, 5 mA; in pulsed waveform, RMS varies with frequency). There was a main effect of stimulation waveforms on AT; F (2, 60)=133.44, p<0.01. Temperature increases (0 to ˜0.4° C.) across frequencies for symmetric biphasic pulsed waveform were significant (p<0.01); however for true square and sinusoidal waveforms, ΔT did not increase significantly across frequencies (p>0.01). Temperature rises appeared to reflect the increase in duty cycle and RMS only for the symmetric biphasic pulsed waveform. Conversely, significantly higher temperatures were measured overall at the 5 mA peak intensity for sinusoid and square waveform compared to the pulsed waveform—reflecting the 100% duty cycles and therefore higher RMS values of the sinusoid and square waveforms. The bioheat model allows titration of duty cycle to a optimal range.

Computational FEM predictions of the phantom using the experimental lead and waveforms were well correlated with experimental temperature increases measurement at varied saline conductivities ((R²=0.24, F (1,40)=12.20, p<0.01, 1.62 S/m; R²=0.26, F (1, 40)=13.70, p<0.01, 0.47 S/m; R²=0.84, F (1,30)=201.84, p<0.01, 0.0047 S/m). Computationally predicted and measured temperature increases were strongly correlated across different RMS stimulation intensities (R²=0.86, F (1, 27)=167.39, p<0.01(FIG. 1D1a)). Accordingly, a strong association between ΔTs were established along radial direction away from the experimental SCS lead; R²=0.96, F (1, 21)=495.59, p<0.01.

Computational Model of Heating by SCS: Influence of Waveform with Standard Tissue Parameters

Using a FEM bio-heat computational models of human spinal cord stimulation, tissue temperature increases were predicted under varied stimulation parameters (Table 1, FIG. 22) for passive heating and active conditions initially using “standard” tissue parameters (see Methods). Six representative SCS waveforms were simulated, with selected frequency and duty cycle (corresponding Pulse Compression Factor noted in table; see Discussion), each with varied peak intensity from 1 to 5 mA (corresponding resultant RMS noted in table). For each waveform and intensity, there is tabulated the maximum ΔT around the SCS clinical lead (E3 contact), at the proximal surface of the dorsal root to the SCS lead (˜1 mm lateral to the stimulating lead), and at the surface of spinal cord (˜3.5 mm radial to the stimulating lead).

From this analysis, several important observations emerge. Heating under the standard active model (which includes blood perfusion (BPer) and metabolic heat generation (MHG)) was lower than the standard passive model (where BPer and MHG were absent). Maximum temperature increases were generated around the SCS clinical lead (the epidural fat). Temperature increases were relatively higher for waveforms with a higher Pulse Compression Factor. Both active and passive heating increased with stimulation RMS, and so with intensity or Pulse Compression Factor, in a super-linear manner (e.g. doubling stimulation intensity or Pulse Compression Factor doubles RMS and results in a >2-fold increase in temperature). While relative temperature increases were more sensitive to intensity than Pulse Compression Factor, the highest temperature increase were predicted under high Pulse Compression (e.g. the 10 KHz waveform). For example, using a conventional 50 Hz waveform (PCF: 1.0), temperature at the spinal cord (SC) increased <0.05° C. even at 5 mA peak (RMS: 0.71) while using a 10 KHz waveform (PCF: 6.32) temperature at the spinal cord (SC) increased ˜1° C. at 5 mA peak (RMS: 4.47). Conventional waveforms and high rate waveforms may thus be used in distinct phases of SCS.

Dependence of temperature increase on RMS (and so Intensity or Pulse Compression Factor) was modeled assuming a power law relationship, which results in a linear log-log dependence (see Methods). Surprisingly, and despite the complexity of the standard tissue model, this fit sufficiently and reliability predicted temperature increases. Slope (β) approached 2 (i.e. temperature increasing with the square of RMS)—a super-linear (β>1) sensitivity of temperature to RMS. The proportionality constant (A) increased across fat (Lead), Spinal Cord, and Root compartments, all relativity higher in the passive versus active tissue model.

Computational Model of SCS: Parameter Sensitivity Analysis with Fixed Waveforms

Living tissue possess complex thermo-electrical properties and these properties are tissue specific. In the active model, the sensitivity of SCS temperature to tissue properties was predicted by halving or doubling the thermal and/or electrical conductivity (from the standard model) of each tissue compartment. At 3.13 mA RMS (as for a 10 KHz SCS waveform with 3.5 mA peak), a significant change in predicted temperature as >0.03° C. and >8% from the standard model was considered. No simulated changes in passive thermal and/or electrical conductivity at any tissue, except epidural fat (eF), produced a significant temperature change at the Lead, Spinal Cord, or Root. However, increases or decreases in epidural fat electrical conductivity significantly decreased or increased temperature across tissue compartments, respectively. The resulting predicted range of temperature increases using waveforms with 3.13 mA RMS were (Passive Model Range; Active Model Range): Lead (1.53-11.57° C.; 1.25-10.77° C.), Spinal Cord (0.42-1.72° C.; 0.18-0.72° C.), and Root (0.17-0.75° C.; 0.04-0.15° C.).

The sensitivity and fit of the power-law function across tissue properties, specifically varying fat electrical (σ) and thermal (k) conductivity (doubling and halving) was considered. In all tissue conditions, the linearity of log-temperature verse log-RMS confirmed a power-law fit, with consistently super-linear sensitivity (β>1). Thus, for each tissue model, temperature could be predicted reliability by simply the corresponding power law function parameters, A and β. In the passive model, β approached 2 across conditions. In the active model β could exceed 2, reflecting variance at low RMS, but not sensitivity at high RMS. The proportionality constant (A) varied more significantly across model parameters and tissue compartments, particularly near the Lead. Here additional electrode designs were modeling where the electrode size and inter-electrode distance are varied. Decreasing inter-electrode distance differentially effects thermopoles as opposed to direct electric field implicated in direct neuromodulation. A key inflection point in differential thermopile and direct activation is at an electrode area of 1 cm2 or less. With this electrode size, a further inflection point is at an inter-electrode distance of 1 mm and again at 0.5 mm. Simulated leads included 4 electrodes each of 1 cm² and an inter-electrode distance of 0.5 mm or 1 mm. A power law relation could be established with a power exceeding 1.8 and 2.5. The resulting predicted range of temperature increases using waveforms with 3.13 mA RMS were (1 mm Passive Model Range; 1 mm Active Model Range; 0.5 mm Passive Model Range; 0.5 mm Active Model Range): Lead (1.73-12° C.; 1-12.7° C.; 1.53-14° C.; 3-12° C.), Spinal Cord (0.532-2.5° C.; 0.6-0.99° C.; 3-15° C.; 4.55-15.87° C.), and Root (0.97-1.44° C.; 1.04-1.16° C.; 1.87-12.12° C.; 1.01-12.11° C.). A lead design with proximal electrodes may thus provide benefit specific to general and control of thermopoles. Or two types of electrodes may be used on a lead, one set designed for direct stimulation and one for generation of thermopoles. A separate key inflection point in differential thermopile and direct activation is at an electrode area of 0.5 cm² or less. With this electrode size, a further inflection point is at an inter-electrode distance of 0.1 mm and again at 0.2 mm. Simulated leads included 4 electrodes each of 0.5 cm2 and an inter-electrode distance of 0.1 mm or 0.2 mm. A power law relation could be established with a power exceeding 1.8 and 2.5. The resulting predicted range of temperature increases using waveforms with 3.13 mA RMS were (0.1 mm Passive Model Range; 0.1 mm Active Model Range; 0.2 mm Passive Model Range; 0.2 mm Active Model Range): Lead (15.6-13° C.; 4.5-16.2° C.; 2.44-16.77° C.; 6.55-15.66° C.), Spinal Cord (0.12-6.58° C.; 0.98-1.82° C.; 6.5-18.56° C.; 6.82-12.56° C.), and Root (1.89-2.66° C.; 1.98-2.2° C.; 2.04-15.92° C.; 5.21-19.31° C.). A lead design with proximal electrodes may thus provide benefit specific to general and control of thermopoles. The role of pulse compression factor increases the power law, so temperature rise in each case. Or two types of electrodes may be used on a lead, one set designed for direct stimulation and one for generation of thermopoles.

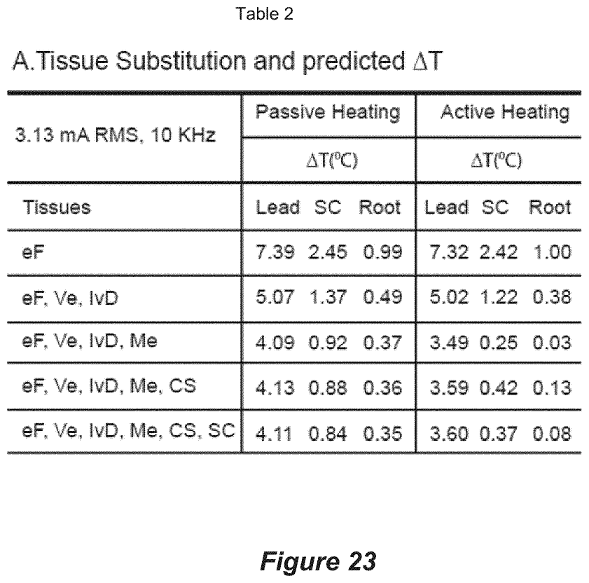

To evaluate the contribution of peripheral spinal tissues on the temperature increases, a series of idealized models staring with uniform epidural fat and then sequentially adding adjacent tissues, under both active and passive model conditions were considered. The order of simulated tissues and predicted maximum temperature increases at locations corresponding to Lead position (“Lead”), Spinal Cord surface (“SC”), and dorsal Root surface (“Root”) are reported for both passive heating and active heating conditions (3.13 mA RMS at 10 KHz; Table 2, FIG. 23). These tissue substitution analysis is not intended to mimic real anatomy but rather elucidate how various tissue compartments shape both temperature field and electric fields. Maximum temperature increases and penetration (from the lead inward) is predicted in the uniform epidural fat model, with a relatively shallow electric field profile. The addition of Soft tissue (St), Vertebrae (Ve), and Intravertebral Disc (IvD) compartments and subsequent reduction of the size of the epidural fat layer result in an incremental reduction in predicted temperatures increases—which is consistent with the notion that fat tissue properties are the most conducive to heating. The relative reduction in temperature between the active and passive models, as well as the reduction in electric field (which is always the same across active and passive models) emphasize these variables can change independently.

Further addition of Meninges (Me) to the model reduced predicted temperature rises notably in both relatively interior (Spinal Cord) and exterior (Lead, Root) regions, indicating that, compared to fat, the Meninges conduct heat away. The reduction in electric field at the Spinal Cord following addition of Meninges (from 165 V/m to 29.27 V/m) was comparable in scale to the temperature decreases in the active model (from 1.22° C. to 0.25° C.) while in the passive model temperature was less sensitive (from 1.37° C. to 0.92° C.), reflecting that the Meninges are vascularized in the active model. Further addition of CSF (CS) decreased predicted temperature rises at the SC and Root for the passive model, increased predicted temperature rises at the Lead for the passive model, and increased temperature in all compartments in the active model. The avascular nature of the CSF layer is overshadowed by its high electrical/thermal conductivity. Finally, addition of Spinal Cord (SC) restores the tissue parameters of standard model. While emphasizing this substitution analysis is abstract, these results reinforce that bio-heat models must include inhomogeneous tissue properties.

Computational Model of Heating by SCS: Role of Time

Time dependent models were implemented. Peak temperature rises in each tissue followed a characteristic time pattern predicted by the bioheat model and reflecting both local tissue properties and surrounding tissue properties. Surrounding tissue properties influences both the rate of heat delivery and/or clearance. The time constant of temperature rise in each tissue was (in minutes, range set but local passive properties) Fat 1-6. Spinal Cord: 3-15; Root: 1-12; Meninges 1-6, white matter 5-34, grey matter 4-32. The time to reach 99% of steady state temperature rise was (in minutes, range set but local passive properties) Fat 2-9. Spinal Cord: 6-20; Root: 2-15; Meninges 2-14, white matter 8-42, grey matter 7-65. As a result of these time constant the step function of temperature change was time limited in minutes and so any change from one stimulation pattern to another would not resolve in temperature fields for minutes. This is in contrast to other forms of direct neuromodulation where changes are relatively instant. Therefore, any adaptive stimulation would have the rate of temperature change limited by the bioheat model of temperature fields which in turn can be power law relationship between each thermopile and local tissue site. The magnitude of the power limited the rate of parameter change as determine by the bio-heat model or by general rules. Finally, a molecular scale model can be added to the bioheat model to implement know kinetics of secondary processes. These can have a time scale of 1-6 hours, days, or weeks including HSP overexpression, reduced NF-kB leading to reduced SGC activation, downregulation of neuronal expression of Csf1, and reduced microglial activation. Neuroimmune processes may also be coupled into the bioheat model based on temperature dependence.

Optimal Deployment of Temperature Sensors

The bioheat simulation indicate the sensitivity of temperature rise to distributed and local tissue anatomy and parameters. To constrain an individualized model, local temperature sensors can be deployed. The bioheat model indicates which locations provide the highest value in constraining the bioheat model while minimize the invasiveness and number or sensors. For a lead or set of leads with a total of X electrodes, X-1 temperature sensors may be used with an inter-sensor distance about half the width of the thermopoles of highest interest. The number of sensors can be reduced as the peak location (but not magnitude) of the thermopoles can be predicted with more accuracy. If the target tissue is full constrained a single thermal sensor can be used to constrain the power law relationship between RMS at each electrode pair and the target tissue. In most SCS deployments, 2 to 6 temperature sensors would provide ideal coverage with positions set by the gradients of the temperature changes. For practical reasons, the position of the sensor may be constrained to on or within the lead. In this case, a temperature sensor between each electrode pair can be used. So, for example a lead with 4 electrodes would include a minimum of 3 temperature sensors.

Discussion

Thermoregulation of CNS temperature depends on a high metabolic activity and both passive (conduction) and active heat exchange (blood flow). Neurostimulation designs for SCS, can challenge this equilibrium in several ways by 1) altering neuronal and so metabolic activity; with 2) changing the cellular microenvironment; 3) changing vascular function as a result of both direct blood vessel stimulation and secondary to microenvironment changes; and 4) depositing of joule heat. In the context of kHz-SCS, the experiments described herein specifically addressed joule heat with the hypothesis that by increased power (pulse compression), kHz-SCS waveforms will superlinearly increase tissue temperature, potentially inducing downstream alterations in tissue function with therapeutic effects in chronic pain. Characteristic clinical responses to kHZ-SCS including as the lack of associated neural sequelae such as paresthesia and the frequency insensitivity of efficacy reconcile well with joule heating, while the delayed time course of effects may be explained by temperature homeostatic responses or heat shock protein regulation of neuroinflammation.

Bioheat SCS Model

FEM bioheat models of the phantom bath, verified by an experiment and of human spinal cord, subjected to a broad parametric sweep (>1400 simulations in this study), are suitable for assessing this hypothesis as they enable predictions as to whether or not SCS may produce temperature rises sufficient to produce biological effects.

Heating from chronic SCS represents an exogenous non-physiological challenge. Temperature increases at the dorsal spinal cord of 0.18-1.72° C. and at the lead in epidural fat of 1.25-11.57° C. under a typical kHz-SCS setting (10 KHz, pulse at 3.5 mA peak; corresponding to 3.13 mA RMS; Table 1, FIG. 22) are predicted. This range depends on epidural fat electrical conductivity; the combination of high current density and low conductivity increases joule heating that is then conducted to other tissues.

The degree of heating is a super-linear function of stimulation RMS power such that kHz-SCS can produce significantly more temperature rise than conventional frequency SCS. Assuming ,β-2 and integrating equation (7) with the power-law relationship (4) yields:

ΔT=0.02*A*1ₚₑₐₖ²*PCF² (8)

where ‘0.02 ’ is the square of RMS₅₀ at 1 mA.

Remarkably, at least across conditions considered here, temperature increases in any tissue inside the spinal canal were fit using a power-law function (equation 8) with all lead positions, electrode configurations, and passive and active tissue properties captured by a single proportionality constant (A). All waveform parameters collapse to Pulse Compression Factor, PCF (Equation 7). This finding has important potential consequences to SCS practice. From a modeling standpoint, this finding dramatically simplifies future efforts to predict temperature changes as part of SCS therapy optimization and programming. Moreover, the super-linear sensitivity to PCF warrant attentions as incremental changes in waveform can spike tissue heating. From a mechanistic standpoint, if temperature increases underpin kHZ-SCS, then waveform power (as captured by PCF) is more important than any single waveform parameter (e.g. frequency, pulse width, shape) in generating effective therapy. However, a heating MoA does not indicate only waveform PCF predicts outcomes as other factors (e.g. electrode placement) influence the proportionality constant (A). Nor does this imply a fixed minimum for stimulation energy (charge, battery consumption) which depends on other factors such as device efficiency and impedance.

Physiological Implications

The nervous system, including the spinal cord, is sensitive to temperature changes. Temperature increases to −44° C. result in brain damage in animal models after 60 minutes, with the temperature threshold for injury decreasing with increased exposure time. In animal models, significant changes in brain excitability have been noted with short-term increases of >2° C., with sensitivity to lower-temperature excepted with long-term temperature increases. Brain temperature increases above 39° C. in ischemic brain injuries increases extracellular excitatory amino acids level, opening of blood-brain barrier, and elevated proteolysis of the neuronal cytoskeleton. A sustained 1-2° C. rise in brain temperature after injury is potentially hazardous. While there are transient changes in temperature during normal function (2-3° C.) a sustained temperature change may produce cumulative and profound changes in brain function. Significant temperature changes in the spinal cord that met or exceeded these thresholds, specifically using kHz frequency waveforms where Pulse Compression increases heat deposition are predicted. These findings are a surprising and an important step toward determining a new heating mechanism for kHz-SCS as well as other relatively high power (kHz frequency) neuromodulation techniques.

Evidence for stimulation acutely changing neuronal firing and metabolism, perfusion, and the extracellular environment is specific (limited) to sub-kHz frequencies for SCS-relevant simulation amplitudes; and so were not modeled here (Qmet and Wb were constant). Starting with kHz-stimulation joule heating, changes in brain function can derive from the acute changes in dynamics (e.g. ion channel gating, neurotransmitter clearance) or a homeostatic molecular response to chronic temperature changes (e.g. heat shock proteins). Slow temperature homeostatic changes provide a plausible explanation for the delayed onset of pain relief by kHz-SCS and suggest specific molecular pathways (MoA) for pain relief including heat shock protein producing downregulation of neuroinflammation. For example, 72-kDa heat shock protein (Hsp70) inhibits activation of the pro-neuroinflammatory transcription factor, nuclear factor-kB in satellite glial cells (NF-kB) (Zheng et al, 2007). Knocking out NF-kB dependent satellite glial cell activation reduces expression of neuronal colony stimulating factor 1 (Csf1), which can potentially reduce the inflammatory response and restore normal function of the spinal pain processing network. Starting with kHz-stimulation joule heating, changes in brain function can derive from the acute changes in dynamics (e.g. ion channel gating, neurotransmitter clearance) or homeostatic molecule response to chronic temperature changes (e.g. heat shock proteins). However, temperature rises must be sufficient to produce beneficial changes without also being enough to cause damage, and as previously noted, the window between physiological baseline (37 C) and temperatures sufficient to produce damage after long exposures (>40 C) may be as low as 3 C, suggesting an energy delivery “sweet spot”. The combination of the observations of long-term changes and a potentially narrow window of beneficial sustained temperature rises provide a plausible explanation for the distinct features of kHz-SCS such as the delayed onset of pain relief by kHz-SCS and a narrow amplitude window over which optimal therapeutic effects occur with burst SCS and suggest a role of temperature rises in the MoA for pain relief (e.g. heat shock protein producing downregulation of neuroinflammation).

Although particular embodiments have been shown and described, the above discussion should not limit the present invention to these embodiments. Various changes and modifications may be made without departing from the spirit and scope of the present invention. Thus, the present invention is intended to cover equivalent embodiments that may fall within the scope of the present invention as defined by the claims.

What is claimed is:

1. A neuromodulation system comprising:
an external device comprising a graphical user interface (GUI) for programming an implantable stimulator device, wherein the implantable stimulator device comprises a plurality of thermodes configured to contact a patient's tissue,
wherein the external device comprises a control circuitry programmed to execute at least a thermopole algorithm,
wherein the thermopole algorithm is configured to:
receive, via the GUI of the external device, one or more inputs indicating one or more prescribed thermopoles in the patient's tissue, and
based on the received one or more inputs, provide the thermal stimulation parameters to the implantable stimulator device for generating the one or more prescribed thermopoles.

2. The neuromodulation system of claim 1, wherein the control circuitry is further programmed to execute at least a bioheat model, wherein the bioheat model is configured to model a thermal response of the patient's tissue to thermal stimulation provided to the patient's tissue by the one or more of the plurality of thermodes and select one or more thermal stimulation parameters for providing the one or more prescribed thermopoles.

3. The neuromodulation system of claim 1, wherein the GUI comprises a representation of the one or more thermodes in relation to the patient's tissue and is configured to represent the one or more prescribed thermopoles.

4. The neuromodulation system of claim 2, wherein the bioheat model comprises a finite element model (FEM) comprising modeled tissue comprising one or more of vertebrae, surrounding soft-tissues, epidural fat, meninges, cerebrospinal fluid, or spinal cord.

5. The neuromodulation system of claim 1, wherein the one or more thermodes comprise one or more thermal elements selected from the group consisting of IR LEDs, low powered lasers, ultrasonic heating elements, piezoelectric heating elements, radio frequency heating elements, and resistive heating elements.

6. The neuromodulation system of claim 1, wherein the one or more thermodes comprise electrodes configured to impart joule heating to the patient's tissue.

7. The neuromodulation system of claim 2, wherein the one or more thermodes comprise electrodes configured to impart joule heating to the patient's tissue and wherein the bioheat model models the thermal response of the patient's tissue to thermal stimulation based on RMS intensity of joule heating imparted at the one or more electrodes.

8. The neuromodulation system of claim 7, wherein the bioheat model models the thermal response of the patient's tissue to thermal stimulation based on a power law function of the RMS intensity corresponding to the formula ΔT=A×RMS^{β}, where ΔT is differences in temperature corresponding to different waveforms, β is a power, and A is a proportionality constant.

9. The neuromodulation system of claim 8, wherein β is a value of 1.4 to 3.5.

10. The neuromodulation system of claim 2, wherein the external device is configured to receive one or more signals from one or more temperature sensors of the implantable stimulation device and wherein the bioheat model is modified based on the one or more signals from the one or more temperature sensors.

11. The neuromodulation system of claim 10, wherein the GUI is configured to represent a temperature map of the patient's tissue based on the one or more signals from the one or more temperature sensors.

12. An implantable stimulator device, comprising:
one or more leads configured for implantation in a patient, the one or more leads comprising a plurality of thermodes, and
a control circuitry programmed to:
cause one or more of the plurality of thermodes to issue thermal stimulation to the patient's tissue, wherein the thermal stimulation is calculated, based on a thermopole algorithm, to elicit a thermopole in the patient's tissue.

13. The implantable stimulator device of claim 12, wherein the one or more thermodes comprise one or more thermal elements selected from the group consisting of IR LEDs, low powered lasers, ultrasonic heating elements, piezoelectric heating elements, radio frequency heating elements, and resistive heating elements.

14. The implantable stimulator device of claim 12, wherein the one or more thermodes comprise a plurality of electrodes configured to impart joule heating to the patient's tissue.

15. The implantable stimulator device of claim 12, wherein the leads further comprise one or more temperature sensors.

16. A method of providing thermal stimulation to a patient's tissue using an implantable stimulator device comprising one or more leads comprising a plurality of thermodes implanted in the patient, the method comprising:
determining one or more desired thermopoles within a target tissue,
using a thermopole algorithm, determining thermal stimulation parameters for two or more of the plurality of thermodes, and
applying thermal stimulation at the one or more of the plurality of thermodes using the determined thermal stimulation parameters.

17. The method of claim 16, wherein the one or more desired thermopoles are determined based at least on a bioheat model.

18. The method of claim 17, wherein the bioheat model comprises a finite element model (FEM) comprising modeled tissue comprising one or more of vertebrae, surrounding soft-tissues, epidural fat, meninges, cerebrospinal fluid, or spinal cord.

19. The method of claim 16, wherein the target tissue is a spinal cord, dorsal root ganglion, or one or more dorsal roots and wherein the one or more leads are implanted in epidural fat.

20. The method of claim 16, further comprising providing electrical neuromodulation in addition to thermal stimulation.
